(19) United States
(12) Patent Application Publication
(10) Pub. No.: US 2020/0129595 A1
(43) Pub. Date: Apr. 30, 2020

(54) STABLE FORMULATIONS OF FIBRONECTIN BASED SCAFFOLD DOMAIN PROTEINS THAT BIND TO MYOSTATIN

(71) Applicant: BRISTOL-MYERS SQUIBB COMPANY, Princeton, NJ (US)

(72) Inventors: Vishal C. NASHINE, Monroe, NJ (US); Rushikesh K. PATEL, New Brunswick, NJ (US)

(21) Appl. No.: 16/607,688

(22) PCT Filed: May 3, 2018
(86) PCT No.: PCT/US2018/030851

§ 371 Date: Oct. 23, 2019

Related U.S. Application Data
(60) Provisional application No. 62/500,649, filed on May 3, 2017.

(51) Int. Cl. A61K 38/39; A61K 47/26; A61K 47/18

(52) U.S. Cl. CPC A61K 38/39; A61K 47/183; A61K 47/26

(57) ABSTRACT

The present invention relates generally to stable liquid formulations comprising polypeptides with ¹⁰Fn3 domains which bind to myostatin and unit dosage forms thereof for administration various routes, including subcutaneous (SC), for treating muscle-wasting and metabolic disorders.

### RELATED APPLICATION INFORMATION

This patent application claims the benefit of U.S. Provisional Patent Application No. 62/500,649, filed May 3, 2017. The entire content of the aforementioned provisional application is incorporated herein by reference.

### FIELD OF THE INVENTION

The present invention relates generally to stable formulations comprising fibronectin-based scaffold domain proteins that bind myostatin, including lyophilized and liquid formulations, for use in therapeutic applications to treat muscle-wasting diseases and metabolic disorders.

### BACKGROUND OF THE INVENTION

Myostatin, also known as growth and differentiation factor-8 (GDF-8), is a member of the transforming growth factor-β (TGF-β) superfamily of secreted growth factors. Myostatin expression is limited primarily to skeletal muscle and adipose tissue, where it has been shown to be a negative regulator of skeletal muscle development (Lee L S, Immunol Endocr Metab Agents Med Chem. 2010; 10:183-194). Both genetic and pharmacological findings indicate that myostatin regulates energy metabolism and that its inhibition can significantly attenuate the progression of metabolic diseases, including obesity and diabetes. For example, myostatin null mice exhibit decreased body fat accumulation (McPherron & Lee, J. JCI 109:595, 2002) when compared with wild type mice of the same age. This reduction in body fat is a manifestation of reduced adipocyte number and size, implicating a significant role of myostatin in adipogenesis as well as in myogenesis. In addition, increases in skeletal muscle mass and strength are associated with metabolic adaptations which positively affect body composition, energy expenditure, glucose homeostasis and insulin requirements.

Over the past two decades, recombinant DNA technology has led to the discovery of a significant number of protein therapeutics. For example, anti-myostatin Adnectins which effectively inhibit myostatin activity in vitro and in vivo have been described (U.S. Pat. Nos. 8,933,199; 8,993,265; 8,853,154; and 9,493,546). These anti-myostatin Adnectins are useful for the treatment of disorders, diseases and conditions for which inhibition of myostatin activity is beneficial, including, for example, muscle wasting diseases, metabolic disorders and conditions resulting in muscle atrophy.

The most conventional route of delivery for protein drugs has been intravenous (IV) administration because of poor bioavailability by most other routes, greater control during clinical administration, and faster pharmaceutical development. For products that require frequent and chronic administration, such as muscle wasting disease and metabolic disorders, the alternate subcutaneous (SC) route of delivery is more appealing. When coupled with pre-filled syringe and autoinjector device technology, SC delivery allows for home administration and improved compliance of administration.

Treatments involving subcutaneous delivery often require development of protein formulations which can be delivered in a small volume (<1.5 ml). For proteins that have a propensity to aggregate achieving stable formulations is a developmental challenge. While the addition of excipients can prevent the formation of aggregates, the number and concentration of excipients required to provide protein stability can lead to an increase in osmolality and/or viscosity that is unsuitable for the rapid administration of small volumes by subcutaneous delivery.

The principles governing protein solubility are more complicated than those for small synthetic molecules, and thus overcoming the protein solubility issue takes different strategies. Operationally, solubility for proteins could be described by the maximum amount of protein in the presence of co-solutes whereby the solution remains visibly clear (i.e., does not show protein precipitates, crystals, or gels). The dependence of protein solubility on ionic strength, salt form, pH, temperature, and certain excipients has been mechanistically explained by changes in bulk water surface tension and protein binding to water and ions versus self-association by Arakawa et al in Theory of protein solubility, Methods of Enzymology, 114:49-77, 1985; Schein in Solubility as a function of protein structure and solvent components, BioTechnology 8(4):308-317, 1990; Jenkins in Three solutions of the protein solubility problem, Protein Science 7(2):376-382, 1998; and others. Binding of proteins to specific excipients or salts influences solubility through changes in protein conformation or masking of certain amino acids involved in self-interaction. Proteins are also preferentially hydrated (and stabilized as more compact conformations) by certain salts, amino acids, and sugars, leading to their altered solubility.

Aggregation which requires bi-molecular collision is expected to be the primary degradation pathway in protein solutions. The relationship of concentration to aggregate formation depends on the size of aggregates as well as the mechanism of association. Protein aggregation may result in covalent (e.g., disulfide-linked) or non-covalent (reversible or irreversible) association. Irreversible aggregation by non-covalent association generally occurs via hydrophobic regions exposed by thermal, mechanical, or chemical processes that alter a protein's native conformation. Protein aggregation may impact protein activity, pharmacokinetics and safety, e.g., due to immunogenicity.

Determining the protein concentration and the type, number and concentration of excipients to obtain stable formulations suitable for subcutaneous delivery remains an empirical exercise due to the labile nature of protein conformation and the propensity to interact with itself, with surfaces, and with specific solutes. For example, whereas wild-type ¹⁰Fn3 is extremely stable and soluble, target-binding variants of ¹⁰Fn3, which contain in the order of 4-31 mutations from the wild-type sequence, vary widely in stability and solubility.

Accordingly, stable pharmaceutical formulations containing fibronectin based molecules which inhibit myostatin are needed for the treatment and/or prevention of disorders or conditions which would benefit from an increase in muscle mass, muscle strength and/or metabolism (e.g., muscular dystrophy, frailty, disuse atrophy and cachexia), disorders associated with muscle wasting (e.g., renal disease, cardiac failure or disease, and liver disease), and metabolic disorders (e.g., Type II diabetes, metabolic syndrome, obesity and osteoarthritis).

### SUMMARY OF THE INVENTION

The present invention provides pharmaceutical formulations containing a concentration of adnectin molecules which inhibit myostatin activity and in which the adnectin molecules remain stable and do not form aggregates or particles. These formulations represent a safe and convenient injectable therapeutic (e.g., once weekly, subcutaneous) useful for increasing muscle mass, muscle strength and/or metabolism in patients in need thereof (e.g., muscle wasting and metabolic disorders).

In one aspect, provided is a stable pharmaceutical formulation comprising (i) at least 10 mg/mL of a polypeptide comprising a fibronectin type III tenth (¹⁰Fn3) domain which binds to myostatin; (ii) a disaccharide at a concentration of at least 5%; (iii) a histidine buffer at a concentration of between about 20 to about 60 mM; and (iv) a pharmaceutically acceptable aqueous carrier, wherein the formulation has a pH range of about 6.5 to about 7.8.

In one embodiment, the formulation comprises a polypeptide comprising a fibronectin type III tenth (¹⁰Fn3) domain which binds to myostatin, wherein at least one loop of the BC, DE, and FG loops of the ¹⁰Fn3 domain has 0, 1, 2, or 3 amino acid substitutions relative to the respective BC, DE, and FG loops of SEQ ID NOs: 5, 6 and 7, respectively. In one embodiment, at least one of the BC, DE, and FG loops of the ¹⁰Fn3 domain has 1 amino acid substitution relative to one loop from the BC, DE, or FG loop of SEQ ID NOs: 5, 6 and 7, respectively. In one embodiment, the ¹⁰Fn3 domain has 1 amino acid substitution relative to the respective BC, DE, or FG loop of SEQ ID NOs: 5, 6 and 7, respectively. In one embodiment, the BC, DE, and FG loops of the ¹⁰Fn3 domain comprise the amino acid sequence of SEQ ID NOs: 5, 6 and 7, respectively.

In a related embodiment, the ¹⁰Fn3 domain comprises an amino acid sequence at least 90%, 91%, 92%, 93%, 94%, 95%, 96%, 97%, 98% or 99% identical to the non-BC, DE, and FG loop regions of SEQ ID NO: 8, 9 or 10. In another related embodiment, the ¹⁰Fn3 domain comprises the amino acid sequence of SEQ ID NO: 8.

In certain embodiments, the polypeptide in the formulation comprises an Fc region. In some embodiments, the Fc is a human IgG. In some embodiments, the Fc is a human IgG1. In certain embodiments, the polypeptide comprises an amino acid sequence at least 90%, 91%, 92%, 93%, 94%, 95%, 96%, 97%, 98% or 99% identical to SEQ ID NO: 78 or SEQ ID NO: 70. In one embodiment, the polypeptide in the formulation comprises SEQ ID NO: 78. In one embodiment, the polypeptide in the formulation consists of SEQ ID NO: 78. In certain embodiments, the polypeptide comprising the ¹⁰Fn3 domain is a dimer.

In some embodiments, the concentration of the polypeptide comprising a fibronectin type III tenth (¹⁰Fn3) domain which binds to myostatin in the formulation is between about 10 mg/mL and 200 mg/mL. In some embodiments, the polypeptide concentration in the formulation is between about 10 mg/mL and about 140 mg/mL, or between about 10 mg/mL and about 85 mg/mL. In other embodiments, the protein concentration of the polypeptide in the formulation is about 10.7 mg/mL, 21.4 mg/mL, 50 mg/mL or 71.4 mg/mL.

In some embodiments, the disaccharide is present at weight (w/w) ratio of at least 5:1 protein to sugar. In some embodiments, the protein:sugar weight ratio is between about 5:1 to 10:1. In some embodiments, the protein:sugar ratio is about 10:1. In some embodiments, the protein:sugar ratio is about 6.75:1.

In some embodiments, the formulation comprises about 5% to about 30%, about 15% to about 25%, or about 20% to about 25% of the disaccharide. In some embodiments, the concentration of the disaccharide is about 150 to about 800 mM or about 300 to about 700 mM. In some embodiments, the concentration of the disaccharide is about 600 mM. In some embodiments, the disaccharide is trehalose. In certain embodiments, the disaccharide is trehalose dihydrate. In some embodiments, the disaccharide is trehalose dihydrate at a concentration of about 600 mM.

In some embodiments, the histidine is present in the formulation at a concentration of at least 20 mM. In some embodiments, the histidine is present at a concentration of between about 20 mM and about 40 mM. In some embodiments, the histidine is present at a concentration of about 20 mM. In some embodiments, the concentration of the histidine in the formulation is about 25 mM. In some embodiments, the histidine is present at a concentration of about 30 mM.

In some embodiments, the pharmaceutical formulation further comprises a surfactant at a concentration between about 0.01% and 0.5%. In some embodiments, the surfactant is polysorbate. In some embodiments, the surfactant is polysorbate 80. In one embodiment, the surfactant is 0.02% PS80.

In some embodiments, the pharmaceutical formulation further comprises a chelator at a concentration between about 0.01 mM and 0.1 mM. Acceptable chelators include, but are not limited to EDTA, DTPA and EGTA. In one embodiment the chelator is DPTA. In one embodiment, the formulation comprises 0.05 mM DTPA.

In some embodiments, the viscosity of the formulation is from about 5 to 20 cps. In some embodiments, the viscosity of the formulation is from about 5 to 15 cps. In some embodiments, the viscosity of the formulation is from about 7 to 12 cps. In some embodiments, the viscosity of the formulation is less than about 8 cps.

In some embodiments, the pharmaceutical formulation is provided in unit dosage form at a volume of between about 0.3 mL to 1 mL. In some embodiments, the pharmaceutical formulation is provided in unit dosage form at a volume of about 0.3 mL, 0.4 mL, 0.5 mL, 0.6 mL, 0.7 mL, 0.8 mL, 0.9 mL or 1.0 mL. In some embodiments, the formulation is provided in unit dosage form of 0.7 mL.

In related embodiments, the unit dosage form comprises between 5 mg and 200 mg of the protein. In some embodiments, the unit dosage comprises about 5 mg, 7.5 mg, 10 mg, 15 mg, 20 mg, 35 mg, 45 mg, 50 mg, 90 mg or 180 mg of the protein.

In some embodiments, the pharmaceutical formulation is formulated for intravenous, intramuscular or subcutaneous injection.

In another aspect, the invention provides a method of attenuating or inhibiting a myostatin-related disease or disorder in a subject by administering an effective amount of a pharmaceutical formulation described above. In some embodiments, the myostatin-related disease or disorder is associated with degeneration or wasting of muscle in the subject. In some embodiments, the myostatin-related disease or disorder is a metabolic disorder.

In certain embodiments, the pharmaceutical formulation is used to treat a conditions selected from Amyotrophic Lateral Sclerosis (ALS), Becker's Muscular Dystrophy (BMD), and Duchenne Muscular Dystrophy (DMD), Spinal Muscular Atrophy (SMA), as well as high-incidence conditions such as sarcopenia and type II diabetes in elderly population. In certain embodiments, the pharmaceutical formulation is used to treat Duchenne Muscular Dystrophy (DMD).

In some embodiments, the subject is a human. In certain embodiments, the subject is a pediatric patient 21 years of age or less. In certain embodiments, the subject is a pediatric patient between about 6 and 12 years of age.

In some embodiments, the polypeptide comprising a fibronectin type III tenth (¹⁰Fn3) domain which binds to myostatin is administered at a dosage of about 5 mg to 200 mg. In some embodiments, the polypeptide comprising a fibronectin type III tenth (¹⁰Fn3) domain which binds to myostatin is administered at a dosage of about 5 mg, 7.5 mg, 10 mg, 15 mg, 20 mg, 35 mg, 45 mg, 50 mg, 90 mg or 180 mg. In certain embodiments, the polypeptide as administered ad a dosage of 7.5, 15, 35, or 50 mg. In some embodiments, the formulation is administered weekly. In some embodiments, the subject is less than about 45 kg and is administered a dosage of about 7.5 mg to about 35 mg. In other embodiments, the subject is more than about 45 kg and is administered a dosage of about 15 mg to about 50 mg.

In a related aspect, the invention provides kits comprising the pharmaceutical formulation described above, and instructions for use.

### BRIEF DESCRIPTION OF THE FIGURES

FIG. 1A depicts the structure of the bivalent polypeptide which binds myostatin. FIG. 1B depicts the amino acid sequence of each polypeptide of the of the bivalent molecule with the amino acid sequence of the Fc portion indicated in bold, with the amino acid of the linker underlined, and with the amino acid sequence of the ¹⁰Fn3 domain indicated in italics.

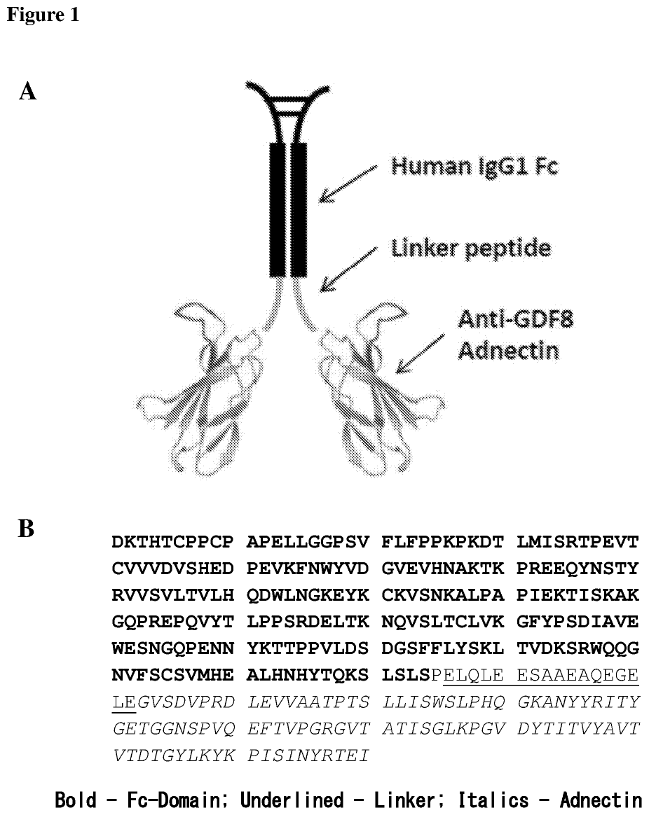

FIG. 2 is a graphic depiction of the percentage of high molecular species in the formulation stored at 5° C. over time at a saccharide concentration of 10% (left panel), and 20% (right panel).

FIG. 3 is a graphic depiction of the percentage of high molecular species in the formulation stored at 25° C. at a saccharide concentration of 10% (upper left panel); stored at 25° C. at a saccharide concentration of 20% (upper right panel); at 35° C. stored at 35° C. at a saccharide concentration of 10% (lower left panel); and stored at 35° C. at a saccharide concentration of 20% (lower right panel).

FIG. 4 is a graphic depiction of the viscosity of formulations containing various concentrations of sucrose or trehalose at different temperatures.

FIG. 5 is a graphic depiction of the percentage of high molecular species after storage for 2 weeks at 25° C. and 35° C. at pH 6.5 or pH 7.0 in presence of sucrose or trehalose.

FIG. 6 is a graphic depiction of viscosity vs. protein concentration in formulations containing 30 mM histidine, 600 mM trehalose dihydrate, 0.05 mM DTPA, 0.02% PS80, pH 7.1.

### DETAILED DESCRIPTION OF THE INVENTION

### I. Definitions

Unless defined otherwise, all technical and scientific terms used herein have the same meaning as commonly understood by the skilled artisan. Although any methods and compositions similar or equivalent to those described herein can be used in practice or testing of the present invention, the preferred methods and compositions are described herein.

The singular form “a,” “an,” and “the” include plural reference unless the context clearly dictates otherwise.

The term “about”, particularly in reference to a given quantity or number, is meant to encompass deviations within plus or minus ten percent (±10%), (e.g., ±5%).

A “stable” formulation or drug product is one in which the anti-myostatin Adnectin therein essentially retains its physical and chemical stability and integrity upon storage. Stability of the anti-myostatin Adnectin molecule formulations can be measured at selected temperatures after selected time periods. For example, an increase in aggregate formation following lyophilization and storage is an indicator for instability of a lyophilized anti-myostatin Adnectin molecule formulation. In addition to aggregate formation, retention of original clarity, color and odor throughout shelf life are indicators utilized to monitor stability of anti-myostatin Adnectin molecule solutions. HMW species are multimers (i.e. tetramers, hexamers, etc.), which have a higher molecular weight than anti-myostatin Adnectin molecule monomers or dimers. Typically a “stable” drug product may be one wherein the increase in aggregation, as measured by an increase in the percentage of high molecular weight species (% HMW), is less than about 5% and preferably less than about 3%, when the formulation is stored at 2-8° C. for one year. Preferably, the manufactured drug product comprises less than about 25% HMW species, preferably less than about 15% HMW species, more preferably less than about 10% HMW species, most preferred less than about 5% HMW species.

“Shelf-life” of a pharmaceutical product, e.g., a protein comprising an anti-myostatin adnectin, is the length of time the product is stored before decomposition occurs. For example, shelf-life may be defined as the time for decomposition of 0.1%, 0.5%, 1%, 5%, or 10% of the product.

The terms “lyophilized” and “freeze-dried” are used interchangeably herein and refer to a material that is dehydrated by first freezing and then reducing the surrounding pressure to allow the frozen water in the material to sublimate.

A “reconstituted” formulation is one which has been prepared by dissolving a lyophilized formulation in an aqueous carrier such that the anti-myostatin Adnectin molecule is dissolved in the reconstituted formulation. The reconstituted formulation is suitable for intravenous administration (IV) or subcutaneous (SC) administration to a patient in need thereof.

An “isotonic” formulation is one which has essentially the same osmotic pressure as human blood. Isotonic formulations will generally have an osmotic pressure from about 250 to 350 mOsmol/KgH2O. The term “hypertonic” is used to describe a formulation with an osmotic pressure above that of human blood. Isotonicity can be measured using a vapor pressure or ice-freezing type osmometer, for example.

The term “buffering agent” refers to one or more components that when added to an aqueous solution is able to protect the solution against variations in pH when adding acid or alkali, or upon dilution with a solvent. Pharmaceutically acceptable buffers include, but are not limited to, histidine, TRIS® (tris (hydroxymethyl) aminomethane), citrate, succinate, glycolate and the like, as described herein.

The term “pKa” refers to the negative logarithm (p) of the ionization (acid dissociation) constant (Kₐ) of an acid which is equal to the pH value at which equal concentrations of the acid and conjugate base forms of a buffer are present (in which half of the acid molecules are ionized). When the p of a buffering agent equals the pH of the solution to be buffered, the buffering system is most effective.

An “acid” is a substance that yields hydrogen ions in aqueous solution. A “pharmaceutically acceptable acid” includes inorganic and organic acids which are nontoxic at the concentration and manner in which they are formulated.

A “base” is a substance that yields hydroxyl ions in aqueous solution. “Pharmaceutically acceptable bases” include inorganic and organic bases which are non-toxic at the concentration and manner in which they are formulated.

A “preservative” is an agent that reduces bacterial action and may be optionally added to the formulations herein. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3pentanol, and m-cresol.

A “surfactant” is a surface active molecule containing both a hydrophobic portion (e.g., alkyl chain) and a hydrophilic portion (e.g., carboxyl and carboxylate groups). Surfactants suitable for use in the formulations of the present invention include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.).

A “drug substance” refers to the starting material utilized in formulation of the final drug product. Typical anti-myostatin adnectin drug substance compositions comprise a protein concentration from 10 mg/mL and 200 mg/mL, pH from 6.6 to 7.6 and % HMW species of <5%.

A “formulated bulk solution” refers to the final formulation prior to filling of the container such as the formulated solution prior to filling the vials for lyophilization, or the formulated solution prior to filling the syringe for IV and/or SC injection.

A “drug product” refers to the final formulation packaged in a container which may be reconstituted before use, such as with a lyophilized drug product; diluted further before use, such as with a liquid drug product; or utilized as is, such as with a SC solution drug product.

“Full-length myostatin” as used herein refers to the full length polypeptide sequence described in McPherron et al. (1997), supra, as well as related full-length polypeptides including allelic variants and interspecies homologs. The term “myostatin” or “mature myostatin” refers to fragments of the biologically active mature myostatin, as well as related polypeptides including allelic variants, splice variants, and fusion peptides and polypeptides. The mature C-terminal protein has been reported to have 100% sequence identity among many species including human, mouse, chicken, porcine, turkey, and rat (Lee et al., PNAS 2001; 98:9306). The sequence for human prepromyostatin is:

(SEQ ID NO: 1)
MQKLQLCVYIYLFMLIVAGPVDLNENSEQKENVEKEGLCNACTWRQNTK
SSRIEAIKIQILSKLRLETAPNISKDVIRQLLPKAPPLRELIDQYDVQRD
DSSDGSLEDDDYHATTETIITMPTESDFLMQVDGKPKCCFFKFSSKIQYN
KVVKAQLWIYLRPVETPTTVFVQILRLIKPMKDGTRYTGIRSLKLDMNPG
TGIWQSIDVKTVLQNWLKQPESNLGIEIKALDENGHDLAVTFPGPGEDGL
NPFLEVKVTDTPKRSRRDFGLDCDEHSTESRCCRYPLTVDFEAFGWDWII
APKRYKANYCSGECEFVFLQKYPHTHLVHQANPRGSAGPCCTPTKMSPIN
MLYFNGKEQIIYGKIPAMVVDRCGCS.

The sequence for human pro-myostatin is:

(SEQ ID NO: 2)
NENSEQKENVEKEGLCNACTWRQNTKSSRIEAIKIQILSKLRLETAPNIS
KDVIRQLLPKAPPLRELIDQYDVQRDDSSDGSLEDDDYHATTETIITMPT
ESDFLMQVDGKPKCCFFKFSSKIQYNKVVKAQLWIYLRPVETPTTVFVQI
LRLIKPMKDGTRYTGIRSLKLDMNPGTGIWQSIDVKTVLQNWLKQPESNL
GIEIKALDENGHDLAVTFPGPGEDGLNPFLEVKVTDTPKRSRRDFGLDCD
EHSTESRCCRYPLTVDFEAFGWDWIIAPKRYKANYCSGECEFVFLQKYPH
THLVHQANPRGSAGPCCTPTKMSPINMLYFNGKEQIIYGKIPAMVVDRCG
CS.

The sequence for mature myostatin (conserved in human, murine, rat, chicken, turkey, dog, horse, and pig) is:

(SEQ ID NO: 3)
DFGLDCDEHSTESRCCRYPLTVDFEAFGWDWIIAPKRYKANYCSGECEF
VFLQKYPHTHLVHQANPRGSAGPCCTPTKMSPINMLYFNGKEQIIYGKI
PAMVVDRCGCS.

As used herein, a “fibronectin based scaffold” or “FBS” protein or moiety refers to proteins or moieties that are based on a fibronectin type III (“Fn3”) repeat. Fibronectin has 18 Fn3 repeats, and while the sequence homology between the repeats is low, they all share a high similarity in tertiary structure. For reviews see Bork et al., Proc. Natl. Acad. Sci. USA, 89(19):8990-8994 (1992); Bork et al., J. Mol. Biol., 242(4):309-320 (1994); Campbell et al., Structure, 2(5):333-337 (1994); Harpez et al., J. Mol. Biol., 238(4):528-539 (1994)). An Fn3 domain is small, monomeric, soluble, and stable. It lacks disulfide bonds and, therefore, is stable under reducing conditions. Fn3 domains comprise, in order from N-terminus to C-terminus, a beta or beta-like strand, A; a loop, AB; a beta or beta-like strand, B; a loop, BC; a beta or beta-like strand, C; a loop, CD; a beta or beta-like strand, D; a loop, DE; a beta or beta-like strand, E; a loop, EF; a beta or beta-like strand, F; a loop, FG; and a beta or beta-like strand, G. The seven antiparallel β-strands are arranged as two beta sheets that form a stable core, while creating two “faces” composed of the loops that connect the beta or beta-like strands. Loops AB, CD, and EF are located at one face (“the south pole”) and loops BC, DE, and FG are located on the opposing face (“the north pole”).

Adnectins are a class of therapeutic FBS proteins with high-affinity and specific target-binding properties that are derived from the tenth human fibronectin type III domain (¹⁰Fn3):

(SEQ ID NO: 4)
VSDVPRDLEVVAATPTSLLISWDAPAVTVRYYRITYGETGGNSPVQEFT
VPGSKSTATISGLKPGVDYTITVYAVTGRGDSPASSKPISINYRT
(BC, DE, and FG loops are underlined).

Accordingly, as used herein, a “¹⁰Fn3 domain” or “¹⁰Fn3 moiety” or “¹⁰Fn3 molecule” refers to wild-type ¹⁰Fn3 and biologically active variants thereof, e.g., biologically active variants that specifically bind to a target, such as a target protein.

A “region” of a ¹⁰Fn3 domain (or moiety or molecule) as used herein refers to either a loop (AB, BC, CD, DE, EF and FG), a β-strand (A, B, C, D, E, F and G), the N-terminus (corresponding to amino acid residues 1-7 of SEQ ID NO: 1), or the C-terminus (corresponding to amino acid residues 93-94 of SEQ ID NO: 1).

A “scaffold region” refers to any non-loop region of a human ¹⁰Fn3 domain. The scaffold region includes the A, B, C, D, E, F and G β-strands as well as the N-terminal region (amino acids corresponding to residues 1-7 of SEQ ID NO: 1) and the C-terminal region (amino acids corresponding to residues 93-94 of SEQ ID NO: 1).

The term “anti-myostatin Adnectin” refers to a protein molecule that binds to and antagonizes myostatin and that comprises at least a one ¹⁰Fn3 domain derived from the human wild-type ¹⁰Fn3 domain (SEQ ID NO: 1). The anti-myostatin Adnectin can further comprise additional protein domains (e.g., an Fc domain), and can also refer to multimer forms of the polypeptide, such as dimers, tetramers and hexamers.

“Polypeptide” as used herein refers to any sequence of two or more amino acids, regardless of length, post-translation modification, or function. “Polypeptide,” “peptide,” and “protein” are used interchangeably herein. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D). The peptides of the invention are proteins derived from the tenth type III domain of fibronectin that have been modified to bind to myostatin and are referred to herein as, “anti-myostatin Adnectin” or “myostatin Adnectin.”

A “polypeptide chain”, as used herein, refers to a polypeptide wherein each of the domains thereof is joined to other domain(s) by peptide bond(s), as opposed to non-covalent interactions or disulfide bonds.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In preferred embodiments, the polypeptide will be purified (1) to greater than 95% by weight of polypeptide as determined by the Lowry method, and most preferably more than 99% by weight, (2) to a degree sufficient to obtain at least residues of N-terminal or internal amino acid sequence by use of a spinning cup sequenator, or (3) to homogeneity by SDS-PAGE under reducing or nonreducing condition using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR™) software. Those skilled in the art can readily determine appropriate parameters for measuring alignment, including any algorithms needed to achieve maximal alignment over the full-length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

As used herein, “conservative substitution” denotes the replacement of amino acid residue by another, without altering the overall conformation and function of the peptide, including, but not limited to, replacement of an amino acid with one having similar properties (such as, for example, polarity, hydrogen bonding potential, acidic, basic, shape, hydrophobic, aromatic, and the like). Exemplary conservative substitutions include those fulfilling the criteria defined for an accepted point mutation in Dayhoff et al., Atlas of Protein Sequence and Structure, 5:345-352 (1978 and Supp.) Examples of conservative substitutions include substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. By “substituted” or “modified” the present invention includes those amino acids that have been altered or modified from naturally occurring amino acids. As such it should be understood that in the context of the present invention a conservative substitution is recognized in the art as a substitution of one amino acid for another amino acid that has similar properties.

As used herein, the term “Adnectin binding site” refers to the site or portion of a protein (e.g., myostatin) that interacts or binds to a particular Adnectin (e.g., as an epitope is recognized by an antibody). Adnectin binding sites can be formed from contiguous amino acids or noncontiguous amino acids juxtaposed by tertiary folding of a protein. Adnectin binding sites formed by contiguous amino acids are typically retained on exposure to denaturing solvents, whereas Adnectin binding sites formed by tertiary folding are typically lost on treatment of denaturing solvents.

The terms “specifically binds,” “specific binding,” “selective binding,” and “selectively binds,” as used interchangeably herein refers to an Adnectin that exhibits affinity for a myostatin, but does not significantly bind (e.g., less than about 10% binding) to a different polypeptide as measured by a technique available in the art such as, but not limited to, Scatchard analysis and/or competitive binding assays (e.g., competition ELISA, BIACORE assay). The term is also applicable where e.g., a binding domain of an Adnectin of the invention is specific for myostatin.

The term “preferentially binds” as used herein refers to the situation in which an Adnectin of the invention binds myostatin at least about 20% greater than it binds a different polypeptide as measured by a technique available in the art such as, but not limited to, Scatchard analysis and/or competitive binding assays (e.g., competition ELISA, BIACORE assay).

As used herein, the term “cross-reactivity” refers to an Adnectin which binds to more than one distinct protein having identical or very similar Adnectin binding sites.

The term “K_{D},” as used herein, is intended to refer to the dissociation equilibrium constant of a particular Adnectin-protein (e.g., myostatin) interaction or the affinity of an Adnectin for a protein (e.g., myostatin), as measured using a surface plasmon resonance assay or a cell binding assay. A “desired K_{D},” as used herein, refers to a K_{D} of an Adnectin that is sufficient for the purposes contemplated. For example, a desired K_{D} may refer to the K_{D} of an Adnectin required to elicit a functional effect in an in vitro assay, e.g., a cell-based luciferase assay.

The term “kₐₛₛ”, as used herein, is intended to refer to the association rate constant for the association of an Adnectin into the Adnectin/protein complex.

The term “k_{diss}”, as used herein, is intended to refer to the dissociation rate constant for the dissociation of an Adnectin from the Adnectin/protein complex.

The term “IC₅₀”, as used herein, refers to the concentration of an Adnectin that inhibits a response, either in an in vitro or an in vivo assay, to a level that is 50% of the maximal inhibitory response, i.e., halfway between the maximal inhibitory response and the untreated response.

The term “myostatin activity” as used herein refers to one or more of growth-regulatory or morphogenetic activities associated with the binding of active myostatin protein to ActRIIb and the subsequent recruitment of Alk4 or Alk5. For example, active myostatin is a negative regulator of skeletal muscle mass. Active myostatin can also modulate the production of muscle-specific enzymes (e.g., creatine kinase), stimulate myoblast proliferation, and modulate preadipocyte differentiation to adipocytes. Myostatin activity can be determined using art-recognized methods, such as those described herein.

The phrases “inhibit myostatin activity” or “antagonize myostatin activity” or “antagonize myostatin” are used interchangeably to refer to the ability of the anti-myostatin Adnectins of the present invention to neutralize or antagonize an activity of myostatin in vivo or in vitro. The terms “inhibit” or “neutralize” as used herein with respect to an activity of an Adnectin of the invention means the ability to substantially antagonize, prohibit, prevent, restrain, slow, disrupt, eliminate, stop, reduce or reverse e.g., progression or severity of that which is being inhibited including, but not limited to, a biological activity or property, a disease or a condition. The inhibition or neutralization is preferably at least about 10%, 20%, 30%, 40%, 50%, 60%, 70%, 80%, 90%, 95% or higher.

For example, an anti-myostatin Adnectin in the pharmaceutical formulation may reduce circulating levels of biologically active myostatin normally found in a vertebrate subject, or a reduction of circulating levels of biologically active myostatin in subjects with disorders that result in elevated circulating levels of myostatin. A reduction of myostatin activity may be determined using in vitro assays, e.g., binding assays, as described herein. Alternatively, a reduction in myostatin activity may result in an increase in body weight, enhanced muscle mass, increased muscle strength, an alteration in the ratio of muscle to fat, an increase in fat-free muscle mass, an increase in the size and/or number of muscle cells, and/or a reduction in body fat content.

The term “PK” is an acronym for “pharmacokinetic” and encompasses properties of a compound including, by way of example, absorption, distribution, metabolism, and elimination by a subject. A “PK modulation protein” or “PK moiety” as used herein refers to any protein, peptide, or moiety that affects the pharmacokinetic properties of a biologically active molecule when fused to or administered together with the biologically active molecule. Examples of a PK modulation protein or PK moiety include PEG, human serum albumin (HSA) binders (as disclosed in U.S. Publication Nos. 2005/0287153 and 2007/0003549, PCT Publication Nos. WO 2009/083804 and WO 2009/133208), human serum albumin, Fc or Fc fragments and variants thereof, and sugars (e.g., sialic acid).

The “half-life” of an amino acid sequence or compound can generally be defined as the time taken for the serum concentration of the polypeptide to be reduced by 50%, in vivo, for example due to degradation of the sequence or compound and/or clearance or sequestration of the sequence or compound by natural mechanisms. The half-life can be determined in any manner known per se, such as by pharmacokinetic analysis. Suitable techniques will be clear to the person skilled in the art, and may for example generally involve the steps of suitably administering to a subject a suitable dose of the amino acid sequence or compound of the invention; collecting blood samples or other samples from the subject at regular intervals; determining the level or concentration of the amino acid sequence or compound of the invention in said blood sample; and calculating, from (a plot of) the data thus obtained, the time until the level or concentration of the amino acid sequence or compound of the invention has been reduced by 50% compared to the initial level upon dosing. Reference is, for example, made to the standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

Half-life can be expressed using parameters such as the t_{1/2}-alpha, t_{1/2}-beta, HL_Lambda_z, and the area under the curve (AUC). In the present specification, an “increase in half-life” refers to an increase in any one of these parameters, any two of these parameters, any three of these parameters or all four of these parameters. An “increase in half-life” in particular refers to an increase in the t_{1/2}-beta, and/or HL_Lambda_z, either with or without an increase in the t_{1/2}-alpha and/or the AUC or both.

The notations “mpk”, “mg/kg”, or “mg per kg” refer to milligrams per kilogram. All notations are used interchangeably throughout the present disclosure.

The terms “individual,” “subject,” and “patient,” used interchangeably herein, refer to an animal, preferably a mammalian (including a nonprimate and a primate) or avian species, including, but not limited to, murines, simians, humans, mammalian farm animals (e.g., bovine, porcine, ovine), mammalian sport animals (e.g., equine), and mammalian pets (e.g., canine and feline); preferably the term refers to humans. The term also refers to avian species, including, but not limited to, chickens and turkeys. In a certain embodiment, the subject, preferably a mammal, preferably a human, is further characterized with a disease or disorder or condition that would benefit from a decreased level or decreased bioactivity of myostatin. In another embodiment the subject, preferably a mammal, preferably a human, is further characterized as being at risk of developing a disorder, disease or condition that would benefit from a decreased level of myostatin or a decreased bioactivity of myostatin.

The term “therapeutically effective amount” refers to at least the minimal dose, but less than a toxic dose, of an agent which is necessary to impart a therapeutic benefit to a subject. For example, a therapeutically effective amount of an anti-myostatin Adnectin of the invention is an amount which in mammals, preferably humans, results in one or more of the following: an increase in muscle volume and/or muscle strength, a decrease in body fat, an increase in insulin sensitivity, or the treatment of conditions wherein the presence of myostatin causes or contributes to an undesirable pathological effect or a decrease in myostatin levels results in a beneficial therapeutic effect.

The term “frail” or “frailty” as used herein refers to a condition that can be characterized by two or more symptoms from weakness, weight loss, slowed mobility, fatigue, low activity levels, poor endurance, and impaired behavioral response to sensory cues. One hallmark of frailty is “sarcopenia,” or the age-related loss of muscle mass.

The term “cachexia” as used herein refers to the condition of accelerated muscle wasting and loss of lean body mass that can result from various diseases.

Various aspects of the present invention are described in further detail in the following subsections.

### II. Myostatin Binding Adnectin Molecules

Anti-myostatin Adnectin molecules that may be used in the formulation provided herein comprise an Fn3 domain derived from the wild-type tenth module of the human fibronectin type III domain (¹⁰Fn3) (SEQ ID NO: 1).

In some embodiments, the anti-myostatin Adnectin in the pharmaceutical formulation comprises the BC, DE, and FG loops as set forth in SEQ ID NOs: 5, 6 and 7, respectively.

In some embodiments, the anti-myostatin Adnectin in the formulation comprises the BC, DE, and FG loops as set forth in SEQ ID NOs: 5, 6 and 7, respectively, wherein the BC loop comprises 1, 2 or 3 amino acid substitutions, such as conservative amino acid substitutions which allow the anti-myostatin Adnectin to maintain binding to myostatin.

In some embodiments, the anti-myostatin Adnectin in the formulation comprises the BC, DE, and FG loops as set forth in SEQ ID NOs: 5, 6 and 7, respectively, wherein at least one loop of the BC, DE, and FG loops of the ¹⁰Fn3 domain has 1 amino acid substitution relative to the respective BC, DE, and FG loops of SEQ ID NOs: 5, 6 and 7.

In some embodiments, the anti-myostatin Adnectin in the formulation comprises the BC, DE, and FG loops as set forth in SEQ ID NOs: 5, 6 and 7, respectively, wherein one loop from the BC, DE, or FG loop of the ¹⁰Fn3 domain has 1 amino acid substitution relative to the respective BC, DE, or FG loop of SEQ ID NOs: 5, 6 and 7.

In some embodiments, the anti-myostatin Adnectin in the formulation comprises the BC, DE, and FG loops as set forth in SEQ ID NOs: 5, 6 or 7, respectively wherein (i) the serine at position 3 of the BC loop (SEQ ID NO: 5) is substituted with an amino acid selected from the group consisting of A, C, D, F, H, I, K, L, N, Q, R, T, V, W, or Y; (ii) the leucine at position 4 of the BC loop (SEQ ID NO: 5) is substituted with an amino acid selected from M or V; (iii) the proline at position 5 of the BC loop (SEQ ID NO: 5) is substituted with an amino acid selected from the group consisting of A, C, D, E, I, K, L, M, N, Q, R, S, T, V, or Y; (vi) the histidine at position 6 of the BC loop (SEQ ID NO: 5) is substituted with an amino acid selected from the group consisting of A, C, D, E, F, G, I, K, L, M, N, Q, R, S, T, V, W, or Y; (vii) the glutamine at position 7 of the BC loop (SEQ ID NO: 5) is substituted with an amino acid selected from the group consisting of A, C, D, E, F, G, H, I, K, L, M, N, P, R, S, T, V, W, or Y; (viii) the glycine at position 8 of the BC loop (SEQ ID NO: 5) is substituted with the amino acid S; (ix) the lysine at position 9 of the BC loop (SEQ ID NO: 5) is substituted with an amino acid selected from the group consisting of A, C, D, E, F, G, H, I, L, M, N, Q, R, S, T, V, W, or Y; (x) the alanine at position 10 of the BC loop (SEQ ID NO: 5) is substituted with an amino acid selected from the group consisting of C, G, L, M, S, or T; or (xi) the asparagine at position 11 of the BC loop (SEQ ID NO: 5) is substituted with an amino acid selected from the group consisting of A, C, F, H, P, Q, R, S, or Y.

In some embodiments, the anti-myostatin Adnectin in the formulation comprises the BC, DE, and FG loops as set forth in SEQ ID NOs: 5, 6 or 7, respectively, wherein (i) the serine at position 3 of the BC loop (SEQ ID NO: 5) is substituted with an amino acid selected from the group consisting of C, F, I, V, W, or Y; (ii) the histidine at position 6 of the BC loop (SEQ ID NO: 6) is substituted with an amino acid selected from the group consisting of C, D, E, F, G, I, K, L, M, N, Q, R, S, T, V, W, or Y; (iii) the lysine at position 9 of the BC loop (SEQ ID NO: 5) is substituted with an amino acid selected from the group consisting of A, C, G, H, I, L, M, N, Q, R, S, V, W, or Y; (iv) the alanine at position 10 of the BC loop (SEQ ID NO: 5) is substituted with an amino acid selected from the group consisting of G, L, M, or S; or (v) the asparagine at position 11 of the BC loop (SEQ ID NO: 5) is substituted with an amino acid selected from the group consisting of C, H, Q, S, or Y.

In some embodiments, the anti-myostatin Adnectin in the formulation comprises the BC, DE, and FG loops as set forth in SEQ ID NOs: 5, 6 or 7, respectively, wherein (i) the serine at position 3 of the BC loop (SEQ ID NO: 5) is substituted with the amino acid F or W; (ii) the histidine at position 6 of the BC loop (SEQ ID NO: 5) is substituted with an amino acid selected from the group consisting of C, F, G, I, K, L, M, N, R, S, T, V, W, or Y; (iii) the glutamine at position 7 of the BC loop (SEQ ID NO: 5) is substituted with an amino acid selected from the group consisting of A, C, E, F, H, I, K, L, M, P, R, S, T, V, or Y; (iii) the lysine at position 9 of the BC loop (SEQ ID NO: 5) is substituted with an amino acid selected from the group consisting of A, C, H, L, M, N, R, V, W, or Y; (iv) the alanine at position 10 of the BC loop (SEQ ID NO: 5) is substituted with the amino acid G or L; or (v) the asparagine at position 11 of the BC loop (SEQ ID NO: 5) is substituted with the amino acid H or Q.

In some embodiments, the anti-myostatin Adnectin in the formulation comprises the BC, DE, and FG loops as set forth in SEQ ID NOs: 5, 6 or 7, respectively, wherein the valine at position 5 of the DE loop (SEQ ID NO: 7) is substituted with an amino acid selected from the group consisting of A, C, D, E, F, I, K, L, M, N, Q, S, or T. In some embodiments, the valine at position 5 of the DE loop (SEQ ID NO: 7) is substituted with an amino acid selected from the group consisting of C, E, I, L, M, Q, or T. In some embodiments, the valine at position 5 of the DE loop (SEQ ID NO: 7) is substituted with an amino acid selected from the group consisting of C, E, I, L, or M.

In some embodiments, the anti-myostatin Adnectin in the formulation comprises the BC, DE, and FG loops as set forth in SEQ ID NOs: 5, 6 or 7, respectively, wherein (i) the valine at position 2 of the FG loop (SEQ ID NO: 7) is substituted with an amino acid selected from the group consisting of A, C, F, I, L, M, Q, T, W, or Y; (iii) the threonine at position 3 of the FG loop (SEQ ID NO: 7) is substituted with an amino acid selected from the group consisting of A, C, F, G, H, I, K, L, M, N, Q, R, S, V, W, or Y; (iv) the aspartic acid at position 4 of the FG loop (SEQ ID NO: 7) is substituted with an amino acid selected from the group consisting of A, C, E, F, G, H, I, K, L, M, N, P, Q, R, S, T, V, W, or Y; (v) the threonine at position 5 of the FG loop (SEQ ID NO: 7) is substituted to with an amino acid selected from the group consisting of A, C, D, E, F, G, H, I, K, L, M, N, P, Q, R, S, V, W, or Y; (vi) the glycine at position 6 of the FG loop (SEQ ID NO: 7) is substituted to with an amino acid selected from the group consisting of A, C, D, E, F, H, I, K, L, M, N, Q, R, S, T, V, W, or Y; (vii) the tyrosine at position 7 of the FG loop (SEQ ID NO: 7) is substituted with an amino acid selected from the group consisting of A, C, F, H, I, L, M, N, P, S, T, V, or W; (viii) the leucine at position 8 of the FG loop (SEQ ID NO: 7) is substituted with an amino acid selected from the group consisting of A, C, E, F, H, I, K, M, N, Q, R, S, T, V, W, or Y; (ix) the lysine at position 9 of the FG loop (SEQ ID NO: 7) is substituted with an amino acid selected from the group consisting of A, C, D, E, F, G, H, I, L, M, N, P, Q, R, S, T, V, W, or Y; (x) the tyrosine at position 10 of the FG loop (SEQ ID NO: 7) is substituted with the amino acid F or W; or (xi) the lysine at position 11 of the FG loop (SEQ ID NO: 7) is substituted with an amino acid selected from the group consisting of A, C, D, E, F, G, H, I, L, M, N, P, Q, R, S, T, V, W, or Y.

In some embodiments, the anti-myostatin Adnectin in the formulation comprises the BC, DE, and FG loops as set forth in SEQ ID NOs: 5, 6 or 7, respectively, wherein (i) the valine at position 2 of the FG loop (SEQ ID NO: 7) is substituted with an amino acid selected from the group consisting of A, C, I, L, or M; (ii) the threonine at position 3 of the FG loop (SEQ ID NO: 7) is substituted with an amino acid selected from the group consisting of C, F, H, I, L, M, Q, R, S, V, W, or Y; (iii) the aspartic acid at position 4 of the FG loop (SEQ ID NO: 7) is substituted with an amino acid selected from the group consisting of A, C, E, F, G, H, I, L, M, N, P, Q, S, T, V, W, or Y; (iv) the threonine at position 5 of the FG loop (SEQ ID NO: 7) is substituted with an amino acid selected from the group consisting of A, C, D, E, F, G, H, I, K, L, M, N, Q, R, S, V, W, or Y; (v) the glycine at position 6 of the FG loop (SEQ ID NO: 7) is substituted with an amino acid selected from the group consisting of A, D, E, F, H, I, L, M, N, Q, S, T, V, W, or Y; (vi) the tyrosine at position 7 of the FG loop (SEQ ID NO: 7) is substituted with an amino acid selected from the group consisting of C, F, I, L, M, P, T, V, or W; (vii) the leucine at position 8 of the FG loop (SEQ ID NO: 7) is substituted with an amino acid selected from the group consisting of C, F, H, I, K, M, N, Q, R, T, V, W, or Y; (viii) the lysine at position 9 of the FG loop (SEQ ID NO: 7) is substituted with an amino acid selected from the group consisting of A, C, E, F, G, I, L, M, N, P, Q, R, S, T, V, W, or Y; (ix) the tyrosine at position 10 of the FG loop (SEQ ID NO: 7) is substituted with the amino acid W; or (x) the lysine at position 11 of the FG loop (SEQ ID NO: 7) is substituted with an amino acid selected from the group consisting of A, C, D, E, G, H, L, M, N, P, Q, R, S, T, or V.

In some embodiments, the anti-myostatin Adnectin in the formulation comprises the BC, DE, and FG loops as set forth in SEQ ID NOs: 5, 6 or 7, respectively, wherein (i) the valine at position 2 of the FG loop (SEQ ID NO: 7) is substituted with the amino acid I; (ii) the threonine at position 3 of the FG loop (SEQ ID NO: 7) is substituted with an amino acid selected from the group consisting of C, F, I, L, M, V, W, or Y; (iii) the aspartic acid at position 4 of the FG loop (SEQ ID NO: 7) is substituted with an amino acid selected from the group consisting of A, C, E, F, G, H, I, L, M, N, Q, S, T, or V; (iv) the threonine at position 5 of the FG loop (SEQ ID NO: 7) is substituted with an amino acid selected from the group consisting of A, C, D, F, G, I, L, M, N, Q, S, V, W, or Y; (v) the glycine at position 6 of the FG loop (SEQ ID NO: 7) is substituted with an amino acid selected from the group consisting of A, S, T, or W; (vi) the tyrosine at position 7 of the FG loop (SEQ ID NO: 7) is substituted with an amino acid selected from the group consisting of F, I, V, or W; (vii) the leucine at position 8 of the FG loop (SEQ ID NO: 7) is substituted with an amino acid selected from the group consisting of F, H, I, M, V, W, or Y; (viii) the lysine at position 9 of the FG loop (SEQ ID NO: 7) is substituted with an amino acid selected from the group consisting of A, C, F, G, I, L, M, T, V, or W; or (x) the lysine at position 11 of the FG loop (SEQ ID NO: 7) is substituted with an amino acid selected from the group consisting of A, G, L, M, P, Q, or R.

In certain embodiments, the anti-myostatin Adnectin comprises an amino acid sequence at least 90%, 95%, 96%, 97%, 98%, 99% or 100% identical to the amino acid sequence set forth in SEQ ID NO: 8:

(SEQ ID NO: 8)
EVVAATPTSLLISWSLPHQGKANYYRITYGETGGNSPVQEFTVPGRGVT
ATISGLKPGVDYTITVYAVTVTDTGYLKYKPIS1NYRT

In some embodiments, the polypeptide in the formulation contains a ¹⁰Fn3 domain which binds myostatin comprising an amino acid sequence at least 90%, 95%, 98%, 99% or 100% identical to the non-BC, DE, and FG loop regions of 8, SEQ ID NO: 9 or SEQ ID NO: 10. For example, in some embodiments, the non-ligand binding sequences of ¹⁰Fn3, i.e., the “¹⁰Fn3 scaffold”, may also be altered provided that the ¹⁰Fn3 retains ligand binding function and/or structural stability. A variety of mutant ¹⁰Fn3 scaffolds have been reported. In some embodiments, one or more of Asp 7, Glu 9, and Asp 23 is replaced by another amino acid, such as, for example, a non-negatively charged amino acid residue (e.g., Asn, Lys, etc.). These mutations have been reported to have the effect of promoting greater stability of the mutant ¹⁰Fn3 at neutral pH as compared to the wild-type form (see, e.g., PCT Publication No. WO 02/04523). A variety of additional alterations in the ¹⁰Fn3 scaffold that are either beneficial or neutral have been disclosed. See, for example, Batori et al., Protein Eng., 15(12):1015-1020 (December 2002); Koide et al., Biochemistry, 40(34):10326-10333 (Aug. 28, 2001).

In certain embodiments, the ¹⁰Fn3 domain of the polypeptide in the formulation comprises SEQ ID NO: 8, SEQ ID NO: 9 or SEQ ID NO: 10. In one embodiment, the ¹⁰Fn3 domain of the polypeptide in the formulation comprises SEQ ID NO: 10.

### Extension Sequences

In certain embodiments, the anti-myostatin Adnectin molecules in the formulation are modified to comprise an N-terminal extension sequence and/or a C-terminal extension sequence. For example, an MG sequence may be placed at the N-terminus of the ¹⁰Fn3 defined by SEQ ID NO: 4. The M will usually be cleaved off, leaving a G at the N-terminus. In some embodiments, the anti-myostatin Adnectin may comprise the amino acid sequence of SEQ ID NO: 8, and an N-terminal extension sequence as shown in Table 1. In addition, an M, G or MG may also be placed N-terminal to any of the N-terminal extensions shown in Table 1. In some embodiments, the anti-myostatin Adnectin in the formulation may be truncated at the threonine corresponding to T94 of SEQ ID NO: 4. Alternatively, C-terminal extensions may be added after the C-terminal residue of SEQ ID NO: 8. Exemplary C-terminal extension sequences are shown in Table 1.

TABLE 1
Summary of N-terminal and C-terminal Extension
Sequences

| SEQ | | | |
|---|---|---|---|
| ID NO | Description | Name | Sequence |
| 11 | Exemplary leader | AdNT1 | MGVSDVPRDL |
| 12 | Exemplary leader | AdNT2 | GVSDVPRDL |
| 13 | Exemplary leader | AdNT3 | VSDVPRDL |
| 14 | Exemplary leader | AdNT4 | SDVPRDL |
| 15 | Exemplary leader | AdNT5 | DVPRDL |
| 16 | Exemplary leader | AdNT6 | VPRDL |
| 17 | Exemplary leader | AdNT7 | PRDL |
| 18 | Exemplary leader | AdNT8 | RDL |
| 19 | Exemplary leader | AdNT9 | DL |
| 20 | Exemplary tail | AdCT1 | EIDKPSQ |
| 21 | Exemplary tail | AdCT2 | EI |
| 22 | Exemplary tail | AdCT3 | EIEPKSS |
| 23 | Exemplary tail | AdCT4 | EIDKPC |
| 24 | Exemplary tail | AdCT5 | EIDKP |
| 25 | Exemplary tail | AdCT6 | EIDK |
| 26 | Exemplary tail | AdCT7 | EIDKPS |
| 27 | Exemplary tail | AdCT8 | EIEKPSQ |
| 28 | Exemplary tail | AdCT9 | EIDKPSQLE |
| 29 | Exemplary tail | AdCT10 | EIEDEDEDEDED |
| 30 | Exemplary tail | AdCT11 | EGSGS |
| 31 | Exemplary tail | AdCT12 | EIDKPCQ |
| 32 | Exemplary tail | AdCT13 | GSGC |
| 33 | Exemplary tail | AdCT14 | EGSGC |
| 34 | Exemplary tail | AdCT15 | EIDKPCQLE |
| 35 | Exemplary tail | AdCT16 | EIDKPSQHHHHHH |
| 36 | Exemplary tail | AdCT17 | GSGCHHHHHH |
| 37 | Exemplary tail | AdCT18 | EGSGCHHHHHH |
| 38 | Tag | T1 | HHHHHH |

In certain embodiments, the C-terminal extension sequences (also called “tails”), comprise E and D residues, and may be between 8 and 50, 10 and 30, 10 and 20, 5 and 10, and 2 and 4 amino acids in length. In some embodiments, tail sequences include ED-based linkers in which the sequence comprises tandem repeats of ED. In exemplary embodiments, the tail sequence comprises 2-10, 2-7, 2-5, 3-10, 3-7, 3-5, 3, 4 or 5 ED repeats. In certain embodiments, the ED-based tail sequences may also include additional amino acid residues, such as, for example: EI, EID, ES, EC, EGS, and EGC. Such sequences are based, in part, on known Adnectin tail sequences, such as EIDKPSQ (SEQ ID NO: 20), in which residues D and K have been removed. In exemplary embodiments, the ED-based tail comprises an E, I or EI residues before the ED repeats.

### Anti-Myostatin Adnectin Immunoglobulin Fc Fusions

In one aspect, provided are formulations containing an anti-myostatin Adnectins comprising fused to an immunoglobulin Fc domain, or a fragment or variant thereof. As used herein, a “functional Fc region” is an Fc domain or fragment thereof which retains the ability to bind FcRn. In some embodiments, a functional Fc region binds to FcRn, but does not possess effector function. The ability of the Fc region or fragment thereof to bind to FcRn can be determined by standard binding assays known in the art. In other embodiments, the Fc region or fragment thereof binds to FcRn and possesses at least one “effector function” of a native Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an anti-myostatin Adnectin) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

In an exemplary embodiment, the Fc domain is derived from an IgG1 subclass, however, other subclasses (e.g., IgG2, IgG3, and IgG4) may also be used. Shown below is the sequence of a human IgG1 immunoglobulin Fc domain:

(SEQ ID NO: 39)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

The core hinge sequence is underlined, and the CH2 and CH3 regions are in regular text. It should be understood that the C-terminal lysine is optional.

The fusion may be formed by attaching an anti-myostatin Adnectin to either end of the Fc molecule, i.e., Fc-anti-myostatin Adnectin or anti-myostatin Adnectin-Fc arrangements. In certain embodiments, the Fc and anti-myostatin Adnectin are fused via a linker. Exemplary linker sequences include GAGGGGSG (SEQ ID NO: 40), EPKSSD (SEQ ID NO: 41), D, ESPKAQASSVPTAQPQAEGLA (SEQ ID NO: 42), ELQLEESAAEAQDGELD (SEQ ID NO: 43), GQPDEPGGS (SEQ ID NO: 44), GGSGSGSGSGSGS (SEQ ID NO: 45), ELQLEESAAEAQEGELE (SEQ ID NO: 46), GSGSG (SEQ ID NO: 47), GSGC (SEQ ID NO: 48), AGGGGSG (SEQ ID NO: 49), GSGS (SEQ ID NO: 50), QPDEPGGS (SEQ ID NO: 51), GSGSGS (SEQ ID NO: 52), TVAAPS (SEQ ID NO: 53), KAGGGGSG (SEQ ID NO: 54), KGSGSGSGSGSGS (SEQ ID NO: 55), KQPDEPGGS (SEQ ID NO: 56), KELQLEESAAEAQDGELD (SEQ ID NO: 57), KTVAAPS (SEQ ID NO: 58), KAGGGGSGG (SEQ ID NO: 59), KGSGSGSGSGSGSG (SEQ ID NO: 60), KQPDEPGGSG (SEQ ID NO: 61), KELQLEESAAEAQDGELDG (SEQ ID NO: 62), KTVAAPSG (SEQ ID NO: 63) AGGGGSGG (SEQ ID NO: 64), AGGGGSG (SEQ ID NO: 65), GSGSGSGSGSGSG (SEQ ID NO: 66), QPDEPGGSG (SEQ ID NO: 67), and TVAAPSG (SEQ ID NO: 68).

In some embodiments, the Fc region used in the anti-myostatin Adnectin fusion comprises the hinge region of an Fc molecule. As used herein, the “hinge” region comprises the core hinge residues spanning positions 1-16 of SEQ ID NO: 39) (DKTHTCPPCPAPELLG; SEQ ID NO: 69) of the IgG1 Fc region.

In certain embodiments, the anti-myostatin Adnectin-Fc fusion in the formulation adopts a multimeric structure (e.g., dimer) owing, in part, to the cysteine residues at positions 6 and 9 of SEQ ID NO: 39 within the hinge region. In other embodiments, the hinge region as used herein, may further include residues derived from the CH1 and CH2 regions that flank the core hinge sequence, as shown in SEQ ID NO: 39. In yet other embodiments, the hinge sequence is GSTHTCPPCPAPELLG (SEQ ID NO: 70).

In some embodiments, the hinge sequence, may include substitutions that confer desirable pharmacokinetic, biophysical, and/or biological properties. Some exemplary hinge sequences include EPKSSDKTHTCPPCPAPELLGGPS (SEQ ID NO: 71; core hinge region underlined), EPKSSDKTHTCPPCPAPELLGGSS (SEQ ID NO 72; core hinge region underlined), EPKSSGSTHTCPPCPAPELLGGSS (SEQ ID NO: 73; core hinge region underlined), DKTHTCPPCPAPELLGGPS (SEQ ID NO: 74; core hinge region underlined), and DKTHTCPPCPAPELLGGSS (SEQ ID NO: 75; core hinge region underlined). In one embodiment, the residue P at position 18 of SEQ ID NO: 39 has been replaced with S to ablate Fc effector function; this replacement is exemplified in hinges having any one of SEQ ID NOs: 72, 73 or 75. In another embodiment, the residues DK at positions 1-2 of SEQ ID NO: 39 have been replaced with GS to remove a potential clip site; this replacement is exemplified in SEQ ID NO: 73. In another embodiment, the C at position 103 of SEQ ID NO: 76, which corresponds to the heavy chain constant region of human IgG1 (i.e., domains CH1-CH3), has been replaced with S to prevent improper cysteine bond formation in the absence of a light chain; this replacement is exemplified in SEQ ID NOs: 71-73.

(SEQ ID NO: 76)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In certain embodiments, an anti-myostatin Adnectin-Fc fusion may have the following configurations: 1) anti-myostatin Adnectin-hinge-Fc or 2) hinge-Fc-anti-myostatin Adnectin. Therefore, any anti-myostatin Adnectin of the present invention can be fused to an Fc region comprising a hinge sequence according to these configurations. In some embodiments, a linker may be used to join the anti-myostatin Adnectin to the hinge-Fc moiety, for example, an exemplary fusion protein may have the configuration anti-myostatin Adnectin-linker-hinge-Fc or hinge-Fc-linker-anti-myostatin Adnectin. Additionally, depending on the system in which the fusion polypeptide is produced, a leader sequence may be placed at the N-terminus of the fusion polypeptide. For example, if the fusion is produced in a mammalian system, a leader sequence such as METDTLLLWVLLLWVPGSTG (SEQ ID NO: 77) may be added to the N-terminus of the fusion molecule. If the fusion is produced in E. coli, the fusion sequence will be preceded by a methionine.

In one embodiment, the formulation contains an Fc-anti-myostatin Adnectin construct comprising the amino acid sequence:

(SEQ ID NO: 78)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPELQLEESAAEAQEGELEGVSDVP
RDLEVVAATPTSLLISWSLPHQGKANYYRITYGETGGNSPVQEFTVPGRG
VTATISGLKPGVDYTITVYAVTVTDTGYLKYKPISINYRTEI.

The hinge region is underlined, the linker is in italics, the leader sequence is in bold, and the anti-myostatin Adnectin sequence is underlined and in italics.

In one embodiment, the formulation contains Fc-anti-myostatin Adnectin construct comprising the amino acid sequence:

(SEQ ID NO: 79)
GVSDVPRDLEVVAATPTSLLISWSLPHQGKANYYRITYGETGGNSPVQEF
TVPGRGVTATISGLKPGVDYTITVYAVTVTDTGYLKYKPISINYRTEIEP
KSSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

The Fc domain comprises the human IgG1 CH2 and CH3 regions as follows:

(SEQ ID NO: 80)
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSP

and the hinge sequence DKTHTCPPCPAPELLG (SEQ ID NO: 69).

### III. Nucleic Acid Molecules and Vectors for Expressing Anti-Myostatin Adnectins

Nucleic acids encoding an anti-myostatin adnectin may be synthesized chemically, enzymatically or recombinantly. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA, 100(2):438-442 (Jan. 21, 2003); Sinclair et al., Protein Expr. Purif., 26(1):96-105 (October 2002); Connell, N. D., Curr. Opin. Biotechnol., 12(5):446-449 (October 2001); Makrides et al., Microbiol. Rev., 60(3):512-538 (September 1996); and Sharp et al., Yeast, 7(7):657-678 (October 1991).

General techniques for nucleic acid manipulation are described for example in Sambrook et al., Molecular Cloning: A Laboratory Manual, Second Edition, Vols. 1-3, Cold Spring Harbor Laboratory Press (1989), or Ausubel, F. et al., Current Protocols in Molecular Biology, Green Publishing and Wiley-Interscience, New York (1987) and periodic updates, herein incorporated by reference. The DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The ability to replicate in a host, usually conferred by an origin of replication, and a selection gene to facilitate recognition of transformants are additionally incorporated.

Accordingly, the anti-myostatin adnectins used in the formulation may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. An exemplary N-terminal leader sequence for production of polypeptides in a mammalian system is: METDTLLLWVLLLWVPGSTG (SEQ ID NO: 81), which is removed by the host cell following expression. For prokaryotic host cells that do not recognize and process a native signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces alpha-factor leaders), or acid phosphatase leader, the C. albicans glucoamylase leader, or the signal described in PCT Publication No. WO 90/13646. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tracycline, (b) complement auxotrophic deficiencies, or (c) supply critical nutrients not available from complex media, e.g., the gene encoding D-alanine racemase for Bacilli.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC® No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC® 20,622 or 38,626) are complemented by known plasmids bearing the Leu2 gene.

Expression and cloning vectors usually contain a promoter that is recognized by the host organism and is operably linked to the nucleic acid encoding the protein of the invention, e.g., a fibronectin-based scaffold protein. Promoters suitable for use with prokaryotic hosts include the phoA promoter, beta-lactamase and lactose promoter systems, alkaline phosphatase, a tryptophan (trp) promoter system, and hybrid promoters such as the tan promoter. However, other known bacterial promoters are suitable. Promoters for use in bacterial systems also will contain a Shine-Dalgarno (S.D.) sequence operably linked to the DNA encoding the protein of the invention.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP Patent Publication No. 73,657 and PCT Publication Nos. WO 2011/124718 and WO 2012/059486. Yeast enhancers also are advantageously used with yeast promoters.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding protein of the invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the peptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of mRNA encoding the protein of the invention. One useful transcription termination component is the bovine growth hormone polyadenylation region. See WO 94/11026 and the expression vector disclosed therein.

The recombinant DNA can also include any type of protein tag sequence that may be useful for purifying the protein. Examples of protein tags include, but are not limited to, a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, New York (1985)), the relevant disclosure of which is hereby incorporated by reference.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (Bio/Technology, 6:47 (1988)). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts.

### IV. Protein Production

Host cells are transformed with the herein-described expression or cloning vectors for protein production and cultured in conventional nutrient media modified as appropriate for inducing promoters, selecting transformants, or amplifying the genes encoding the desired sequences. The host cells used to produce the anti-myostatin adnectins may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma)) are suitable for culturing the host cells. In addition, many of the media described in Ham et al., Meth. Enzymol., 58:44 (1979), Barites et al., Anal. Biochem., 102:255 (1980), U.S. Pat. Nos. 4,767,704, 4,657,866, 4,927,762, 4,560,655, 5,122,469, 6,048,728, 5,672,502, or U.S. Pat. No. RE 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Proteins of the invention can also be produced by chemical synthesis (e.g., by the methods described in Solid Phase Peptide Synthesis, 2nd Edition, The Pierce Chemical Co., Rockford, Ill. (1984)). Modifications to the protein can also be produced by chemical synthesis.

The proteins can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, get filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, or preferably at least 95% pure, and most preferably at least 98% pure. Regardless of the exact numerical value of the purity, the polypeptide is sufficiently pure for use as a pharmaceutical product.

Monomer, dimer and HMW species of anti-myostatin Adnectin molecule may be separated by size exclusion chromatography (SEC). SEC separates molecules based on the molecular size. Separation is achieved by the differential molecular exclusion or inclusion as the molecules migrate along the length of the column. Thus, resolution increases as a function of column length. Anti-myostatin Adnectin molecule samples may be separated using a 2695 Alliance HPLC (Waters, Milford, Mass.) equipped with TSK Gel G3000SWxL (300 mm×7.8 mm, 5 microns) and TSK Gel SWxL (40 mm×6.0 mm, 7 microns) columns (Tosoh Bioscience, Montgomery, Pa.) in tandem. Neat samples with injection weight of 200 μg are separated using a mobile phase consisting of 40 mM NaH2PO4, 60 mM Na2HPO4, 0.1 M Na2SO4, pH 6.8, at a flow rate of 0.5 ml/min. Samples are monitored at an absorbance of 280 nm using Water's 2487 Dual Wavelength detector. Using this system, the HMW species has a retention time of 16.0 min±1.0 min. Each peak is integrated for area under the peak. The % HMW species calculated by dividing the HMW peak area by the total peak area.

### V. Measurement of Anti-Myostatin Adnectin Activity

Binding of an anti-myostatin Adnectin of the invention to a target molecule (e.g., myostatin) may be assessed in terms of equilibrium constants (e.g., dissociation, K_{D}) and in terms of kinetic constants (e.g., on-rate constant, kₒₙ and off-rate constant, k_{off}). Exemplary in vitro and in vivo assays for assessing the binding activity of an anti-myostatin Adnectin in the pharmaceutical formulations provided herein have been previously described (e.g., U.S. Pat. Nos. 8,933,199; 8,993,265; 8,853,154; and 9,493,546) and include, but are not limited to, solution phase methods such as the kinetic exclusion assay (KinExA) (Blake et al., JBC 1996; 271:27677-85; Drake et al., Anal Biochem 2004; 328:35-43), surface plasmon resonance (SPR) with the Biacore system (Uppsala, Sweden) (Welford et al., Opt. Quant. Elect 1991; 23:1; Morton and Myszka, Methods in Enzymology 1998; 295:268), homogeneous time resolved fluorescence (HTRF) assays (Newton et al., J Biomol Screen 2008; 13:674-82; Patel et al., Assay Drug Dev Technol 2008; 6:55-68), and using a Biacore surface plasmon resonance system (Biacore, Inc.). It should be understood that the assays described herein above are exemplary, and that any method known in the art for determining the binding affinity between proteins (e.g., fluorescence based-transfer (FRET), enzyme-linked immunosorbent assay, and competitive binding assays (e.g., radioimmunoassays)) can be used to assess the binding affinities of the anti-myostatin Adnectins of the invention.

The ability of anti-myostatin Adnectins to antagonize myostatin activity can be readily determined using various in vitro assays. Preferably, the assays are high-throughput assays that allow for screening multiple candidate Adnectins simultaneously. In some embodiments, the antagonist effects of anti-myostatin Adnectins on myostatin activity can be determined in cell-based activin responsive element (ARE)-luciferase reporter assays, as described in Example 3. In certain embodiments, the anti-myostatin Adnectins of the invention decrease myostatin-induced ARE-luciferase activity by at least 10%, at least 20%, at least 30%, at least 40%, at least 50%, at least 60%, at least 70%, at least 80%, at least 90% or more relative to a control upon co-incubating myostatin with an anti-myostatin Adnectin prior to stimulating cells with the mixture. An exemplary control reaction involves treating cells with myostatin alone or myostatin preincubated with an excess of a benchmark myostatin inhibitor such as Human Activin RIIB Fc Chimera (R&D Systems) or ActRIIb-Fc as described in Morrison et al. (Experimental Neurology 2009; 217:258-68). In other embodiments, the anti-myostatin Adnectins of the invention inhibit ARE-luciferase reporter activity with an IC50 of 500 nM or less, 400 nM or less, 300 nM or less, 200 nM or less, 100 nM or less, 50 nM or less, 10 nM or less, 5 nM or less, 1 nM, 0.5 nM or less, 0.4 nM or less, 0.3 nM or less, 0.2 nM or less, or 0.10 nM or less, as described in Example 3.

In other embodiments, the antagonistic effects of anti-myostatin Adnectins on myostatin activity can be determined by measuring the extent of SMAD phosphorylation in myostatin-treated cells, as described in U.S. Pat. Nos. 8,933,199; 8,993,265; 8,853,154; and 9,493,546. An exemplary control reaction involves treating cells with myostatin alone or myostatin preincubated with an excess of a benchmark myostatin inhibitor such as Human Activin RIIB Fc Chimera (R&D Systems) or ActRIIb-Fc as described in Morrison et al. (Experimental Neurology 2009; 217:258-68). In some embodiments, the anti-myostatin Adnectins of the invention inhibit SMAD phosphorylation with an IC50 of 1 nM or less, 0.8 nM or less, 0.6 nM or less, 0.4 nM or less, 0.3 nM or less, 0.2 nM or less, or 0.1 nM or less in a 12-point or 4-point inhibition response, as described in Example 5. In other embodiments, the anti-myostatin Adnectins of the invention at 10 nM inhibit SMAD phosphorylation by myostatin by at least 50%, at least 60%, at least 70%, at least 80%, at least 90%, at least 91%, at least 92%, at least 93%, at least 94%, at least 95%, at least 96%, at least 97%, or at least 98% or more.

Additionally, several in vitro model systems are known which use cells, tissue culture and histological methods for studying motor neuron disease. For example, a rat spinal cord organotypic slice subjected to glutamate excitotoxicity is useful as a model system to test the effectiveness of anti-myostatin Adnectins in preventing motor neuron degeneration. Corse et al., Neurobiol. Dis. (1999) 6:335 346. For a discussion of in vitro systems for use in studying ALS, see, e.g., Bar, P. R., Eur. J. Pharmacol. (2000) 405:285 295; Silani et al., J. Neurol. (2000) 247 Suppl 1:128 36; Martin et al., Int. J. Mol. Med. (2000) 5:3 13.

It should be understood that the assays described herein are exemplary, and that any method known in the art that can serve as a readout for myostatin activity are suitable for use for testing the myostatin antagonizing effects of the anti-myostatin Adnectins of the invention (e.g., real-time RT-PCR of mRNAs of SMAD target genes (e.g., Smad 7; Ciarmela et al., Journal of Clinical Endocrinology & Metabolism 2011; 96; 755-65) or mRNAs of ARE-containing genes).

### VI. Formulations

For subcutaneous administration, a dosage which delivers the desired protein concentrations in a small volume (<1.5 mL) is desired. Accordingly, the SC formulations provided herein comprises the anti-myostatin adnectin at a protein concentration of at least 10 mg/mL in combination with a disaccharide at stabilizing levels and a buffering agent, in an aqueous carrier. In some embodiments, the protein concentration of the anti-myostatin adnectin in the formulation is between about 10 mg/mL and 200 mg/mL. In some embodiments, the protein concentration of the anti-myostatin adnectin in the formulation is between about 10 mg/mL and 140 mg/mL

In some embodiments, the protein concentration of the anti-myostatin adnectin in the formulation is least about 10 mg/mL, 15 mg/mL, 20 mg/mL, 25 mg/mL, 30 mg/mL, 35 mg/mL, 40 mg/mL, 45 mg/mL, 50, mg/mL, 60 mg/mL, 65 mg/mL, 70 mg/mL, 75 mg/mL, 80 mg/mL, 85 mg/mL, 90 mg/mL, 95 mg/mL or higher. In certain embodiments, protein concentration of anti-myostatin adnectin in the formulation is at least about 110 mg/mL, 115 mg/mL, 120 mg/mL, 125 mg/mL, 130 mg/mL, 135 mg/mL, 140, mg/mL or 145, mg/mL. In certain embodiments, the protein concentration of the anti-myostatin adnectin in the formulation is 10.7 mg/mL, 21.4 mg/mL, 50.0 mg/mL or 71.4 mg/mL.

The stabilizing sugar in the formulation is a disaccharide in a weight (w/w) ratio of at least 5:1 protein to sugar. In some embodiments, the protein:sugar weight ratio is between about 5:1 to 10:1. In some embodiments, the protein:sugar ratio is about 6:1, 7:1, 8:1, 9:1 or 10:1. In some embodiments, the protein:sugar ratio is about 6.75:1.

In some embodiments, the formulation comprises about 5% to about 30% of the disaccharide. In some embodiments, the formulation comprises about 10% to about 28% of the disaccharide. In some embodiments, the formulation comprises about 15% to about 25% of the disaccharide. In some embodiments, the formulation comprises about 20% to about 25% of the disaccharide. In some embodiments, the formulation comprises about 18%, 19%, 20%, 21%, 22%, 23%, 24% or about 25% of the disaccharide.

In some embodiments, the concentration of the sugar in the formulation is about 150 mM to about 800 mM. In some embodiments, the concentration of the sugar in the formulation is about 300 to about 700 mM. In other embodiments, the concentration of the sugar in the formulation is about 150 mM, about 200 mM, about 250 mM, about 300 mM, about 350 mM, about 400 mM, about 450 mM, about 500 mM, about 550 mM, about 575 mM, about 600, about 625 mM, about 650 mM, about 675 mM or about 700 mM.

In some embodiments, the disaccharide is trehalose. In some embodiments, the formulation comprises about 5 to about 30% trehalose. In some embodiments, the formulation comprises about 10% to about 28% trehalose. In some embodiments, the formulation comprises about 15% to about 25% trehalose. In some embodiments, the formulation comprises about 20% to about 25% trehalose. In some embodiments, the formulation comprises about 18%, 19%, 20%, 21%, 22%, 23%, 24% or about 25% trehalose. In one embodiment, the formulation comprises 22% trehalose. In another embodiment, the formulation comprises 23% trehalose.

In some embodiments, the disaccharide is trehalose dihydrate. In some embodiments, concentration of trehalose dihydrate in the formulation is about 150 mM to about 800 mM. In some embodiments, the concentration of the trehalose dihydrate in the formulation is about 300 to about 700 mM. In other embodiments, the concentration of the trehalose dihydrate in the formulation is about 150 mM, about 200 mM, about 250 mM, about 300 mM, about 350 mM, about 400 mM, about 450 mM, about 500 mM, about 550 mM, about 575 mM, about 600, about 625 mM, about 650 mM, about 675 mM or about 700 mM. In one embodiment, the concentration of trehalose dihydrate in the formulation is 600 nM.

The stabilizing sugar in the formulation is employed in an amount no greater than that which may result in a viscosity undesirable or unsuitable for administration via SC syringe. In some embodiments, the viscosity of the formulation is from about 5 to 20 cps. In some embodiments, the viscosity of the formulation is from about 7 to 12 cps. In some embodiments, the viscosity is about 7-10 cps. In some embodiments, the viscosity of the formulation is less than 8 cps.

The buffering agent in the formulation is present in an amount of at least 20 mM, and is preferably between about 20 mM and about 40 mM. In some embodiments, the buffering agent is histidine at a concentration of about 20 mM, about 25 mM, about 30 mM or about 35 mM. In one embodiment, the formulation comprises about 30 mM histidine.

The pH of the formulation is maintained at a range from about 6.5 to about 7.8. In certain embodiments, the pH is maintained at a range from about pH from 6.6 to 7.6. In certain embodiments, the pH of the formulation is about 6.8 to 7.4. In certain embodiments, the pH of the formulation is about 7.0 to 7.3. In some embodiments, the pH of the formulation is 6.9, 7.0, 7.1, 7.2 or 7.3. In some embodiments, the pH of the formulation is about 7.1.

The aqueous carrier used in the formulations herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

The formulations may further comprise a surfactant to further reduce the formation of visible particulates. Preferred surfactants include poloxamer and polysorbate at a concentration of between about 0.01% and 0.5%. In some embodiments, the concentration of the surfactant is between about 0.02% and about 0.1%. In one embodiment, the surfactant is poloxamer 188. In some embodiments, the surfactant is polysorbate 20 or polysorbate 80. In one embodiment, the surfactant is polysorbate 80.

The formulations may further comprise a chelator at a concentration between about 0.01 mM and about 0.5 mM, preferably, between about 0.05 mM and 0.2 mM. Preferred chelators include, but are not limited to DPTA, EDTA and EGTA. In one embodiment, the chelator in the formulation is DPTA at a concentration of about 0.05 mM.

A preservative may be optionally added to the formulations herein to reduce bacterial action. The addition of a preservative may, for example, facilitate the production of a multi-use (multiple-dose) formulation.

In some embodiments, the formulation provided herein comprises:

(i) about 10-140 mg/mL of an anti-myostatin adnectin;

(ii) about 5-25% trehalose dihydrate; and

(iii) about 20-30 mM histidine,

wherein the pH of the formulation is about 6.8 to 7.3.

In some embodiments, the formulation provided herein consists essentially of:

(i) about 10-140 mg/mL of an anti-myostatin adnectin;

(ii) about 5-25% trehalose dihydrate; and

(iii) about 20-30 mM histidine,

wherein the pH of the formulation is about 6.8 to 7.3.

In certain embodiments, the formulation provided herein comprises:

(i) about 10-140 mg/mL of an anti-myostatin adnectin;

(ii) about 5-25% trehalose dihydrate;

(iii) about 20-30 mM histidine;

(iv) about 0.02-0.06 mM DTPA; and

(v) about 0.01-0.05% polysorbate 80

wherein the pH of the formulation is about 6.8 to 7.3.

In certain embodiments, the formulation provided herein consists essentially of:

(i) about 10-140 mg/mL of an anti-myostatin adnectin;

(ii) about 5-25% trehalose dihydrate;

(iii) about 20-30 mM histidine;

(iv) about 0.02-0.06 mM DTPA; and

(v) about 0.01-0.05% polysorbate 80

wherein the pH of the formulation is about 6.8 to 7.3.

In some embodiments, the formulation comprises:

(i) about 10-75 mg/mL of an anti-myostatin adnectin;

(ii) about 600 mM trehalose dihydrate; and

(iii) 25-30 mM histidine,

wherein the pH of the formulation is about 7.0 to 7.3.

In some embodiments, the formulation consists essentially of:

(i) about 10-75 mg/mL of an anti-myostatin adnectin;

(ii) about 600 mM trehalose dihydrate; and

(iii) 25-30 mM histidine,

wherein the pH of the formulation is about 7.0 to 7.3.

In some embodiments, the formulation comprises:

(i) about 10-75 mg/mL of an anti-myostatin adnectin;

(ii) about 600 mM trehalose dihydrate;

(iii) 25-30 mM histidine;

(iv) about 0.02-0.06 mM DTPA; and

(v) about 0.01-0.05% polysorbate 80,

wherein the pH of the formulation is about 7.0 to 7.3.

In some embodiments, the formulation consists essentially of:

(i) about 10-75 mg/mL of an anti-myostatin adnectin;

(ii) about 600 mM trehalose dihydrate;

(iii) 25-30 mM histidine;

(iv) about 0.02-0.06 mM DTPA; and

(v) about 0.01-0.05% polysorbate 80,

wherein the pH of the formulation is about 7.0 to 7.3.

In some embodiments, the formulation comprises

(i) about 10-75 mg/mL of an anti-myostatin adnectin;

(ii) about 600 mM trehalose dihydrate;

(iii) about 30 mM histidine;

(iv) about 0.05 mM DTPA; and

(v) about 0.02% polysorbate 80,

wherein the pH of the formulation is about 7.1.

In some embodiment, the formulation consists essentially of:

(i) about 10-75 mg/mL of an anti-myostatin adnectin;

(ii) about 600 mM trehalose dihydrate;

(iii) about 30 mM histidine;

(iv) about 0.05 mM DTPA; and

(v) about 0.02% polysorbate 80,

wherein the pH of the formulation is about 7.1.

In one embodiment, the formulation comprises or consists essentially of:

(i) about 10.7 mg/mL of an anti-myostatin adnectin;

(ii) about 600 mM trehalose dihydrate;

(iii) about 30 mM histidine;

(iv) about 0.05 mM DTPA; and

(v) about 0.02% polysorbate 80,

wherein the pH of the formulation is about 7.1.

In one embodiment, the formulation comprises or consists essentially of:

(i) about 21.4 mg/mL of an anti-myostatin adnectin;

(ii) about 600 mM trehalose dihydrate;

(iii) about 30 mM histidine;

(iv) about 0.05 mM DTPA; and

(v) about 0.02% polysorbate 80,

wherein the pH of the formulation is about 7.1.

In one embodiment, the formulation comprises or consists essentially of:

(i) about 50 mg/mL of an anti-myostatin adnectin;

(ii) about 600 mM trehalose dihydrate;

(iii) about 30 mM histidine;

(iv) about 0.05 mM DTPA; and

(v) about 0.02% polysorbate 80,

wherein the pH of the formulation is about 7.1.

In one embodiment, the formulation comprises or consists essentially of:

(i) about 71.4 mg/mL of an anti-myostatin adnectin;

(ii) about 600 mM trehalose dihydrate;

(iii) about 30 mM histidine;

(iv) about 0.05 mM DTPA; and

(v) about 0.02% polysorbate 80,

wherein the pH of the formulation is about 7.1.

The recommended storage condition for the liquid formulation is from 2-8° C., with a recommended shelf life of at least 12 months.

In order to ensure efficacy and safety during the time course of the shelf life of pharmaceutical compositions, the composition is stability tested. Typically, the stability tests include but are not limited to tests regarding identity, purity and potency of the composition. The stability is tested both at the intended storage temperature and at elevated temperature or temperatures. Purity tests may include but are not limited to SDS-PAGE, CE-SDS, isoelectrofocusing, immunoelectrophoresis, Western blot, reversed-phase chromatography, Size-exclusion chromatograhy (SEC), ion exchange and affinity chromatography. Other tests may include, but are not limited to: visual appearance such as colour and transparency, particulates, pH, protein concentration measurement, moisture and reconstitution time.

The degradation profile regarding, in particular, purity and potency, during the stability time course is intimately coupled to the composition and/or the formulation of the pharmaceutical product. In particular, proper choice of formulation may significantly change the degradation profile. Typical degradation profiles for protein molecules products derived from FBS includes the formation of covalent and non-covalent high molecular weight aggregates, fragments, deamidation and oxidation products. Particularly, de-amidation and oxidation products as well as other acidic species usually develop during the time course of the stability testing. In some cases the acidic species limits the acceptable shelf life of the pharmaceutical composition. The formation of acidic species due to, for example, deamidation, can be tested by, e.g., imaged capillary isoelectrofocusing (icIEF). In other cases the formation of high molecular weight aggregates limits the acceptable shelf life of the pharmaceutical composition. The formation of aggregates may be tested by for example SEC (size exclusion chromatography), DLS, MFI, SDS-PAGE or CE-SDS.

For example, an anti-myostatin Adnectin formulation with pharmaceutically acceptable stability can be one wherein, when stored at a temperature of about 5±3° C. or 25±2° C. for a period of least about 3 months, preferably about 6 months, and more preferably about 12 months or longer, such as 18 months or longer, such as for at least 24 or even 36 months, the percentage of aggregates is less than about 10%, preferably less than about 5%, more preferably less than about 2%, when determined using SEC analysis. Additionally or alternatively, a stable anti-myostatin Adnectin formulation of the invention can be one wherein, when stored at a temperature of about 5±3° C. or 25±2° C. for a period of least about 3 months, preferably about 6 months, and more preferably about 12 months or longer, the changes of main isoform are less than 15%, preferably less than 10%, more preferably less than 8%, most preferably less than 5%, when determined using icIEF analysis.

The Examples provided herein describe stability studies of SC formulations demonstrating that the stability of the anti-myostatin adnectin molecule in the SC formulation is enhanced in the presence of trehalose over that of sucrose. Stabilization by trehalose was better at protein:trehalose ratios of less than 10:1. Based on these studies, trehalose was selected as the stabilizer at a ratio that provides optimum stability without resulting in a SC solution with excessive hypertonicity or viscosity.

It was also observed that formulations containing histidine as a buffering agent at a pH of 7.0-7.1 exhibited better stability (i.e., lower % HMW) than phosphate buffer after storage at 25° C. and 37° C. (data not shown). This observation was particularly surprising in view of the pKa of histidine (pka=6.0), and appears to be based at least in part on the unexpected finding that the anti-myostatin adnectin has an inherent buffering capacity. This allows the production of formulations away from the pKa of the buffer while leveraging the buffering capacity of the protein to attain the pH stability needed during the manufacture and shelf-life of the product.

### VII. Preparation of the SC Formulation

The manufacturing process developed for SC formulations typically involves compounding with sugar, chelating agent and surfactant, followed by aseptic sterile filtration and filling into vials or syringes, optionally preceded by diafiltration (buffer exchange) and concentration of drug substance using an ultrafiltration unit. The protein purification is the first stage after production in the fermentation bioreactor. The protein is purified using multiple column and filtration steps and concentrated using tangential flow filtration into the formulation buffer. The concentrated drug substance is diluted with the formulation buffer at the target concentrations and this solution is sterile filtered and filled into sterile vials/syringes for patient use. One skilled in the art would be aware of the need to overfill the container so as to compensate for vial, needle, syringe hold-up during preparation and injection. For example, a 5-10% overage of drug product is incorporated into each vial of liquid formulation to account for withdrawal losses and guarantee that required dose (label claim) of drug product can be withdrawn from the vial.

Preparation of unit dosage forms for the formulation comprising the polypeptide comprising the ¹⁰Fn3 domain which binds myostatin (also referred interchangeably herein as “anti-myostatin adnectin”) syringes involves protein production in a recombinant cell line, purification vial multiple column steps, concentration and buffer exchange into formulation buffer using tangential flow filtration. The concentrated protein for the tangential flow filtration is further processed by dilution with formulation buffer to the target protein concentrations and the diluted product, after filtration, is filled into 1 mL syringes (e.g., insulin syringe, tuberculin syringe, BioPak syringe, NeoPak syringe). In one embodiment, the syringes are then equipped with an UltraSafe Passive needle guard.

The unit dosage form of the formulation typically contains about 0.3 to 1.5 mL of the formulation. In certain embodiments, the unit dosage form contains a volume of 0.3, 0.5, 0.7, 0.8, 1.0, 1.2, 1.4 or 1.4 mL. In certain embodiments, the unit dosage form is provided at a volume of 0.7 mL. In some embodiments, the unit dosage form contains 5-100 mg of the polypeptide comprising the ¹⁰Fn3 domain which binds myostatin. In some embodiments, the unit dosage form comprises 7.5 mg, 15 mg, 35 mg or 50 mg of the anti-myostatin adnectin.

In some embodiments, the formulations are manufactured as disclosed herein and are stored in bulk at −60° C., for example, in 12 L FFTp bags at polypeptide concentration of 85-150 mg/mL. In some embodiments, the formulations are stored at −60° C. at a polypeptide concentration of 85 mg/mL. The bulk formulations are then thawed and diluted to the appropriate polypeptide concentration for preparation of the unit dosage forms. In some embodiments, the polypeptide concentration of the formulation in the unit dosage form is about 10 mg/mL to about 140 mg/mL. In some embodiments, the polypeptide concentration of the formulation in the unit dosage form is about 10 mg/mL to about 75 mg/mL. In certain embodiments, the polypeptide concentration of the formulation in the unit dosage form is 10.7 mg/mL, 20.4 mg/ml, 50 mg/mL or 71.4 mg/mL.

### VIII. Administration

A pharmaceutical formulation comprising an anti-myostatin Adnectin of the present invention can be administered to a subject at risk for or exhibiting pathologies as described herein. The formulations provide herein are particularly useful for peripheral systemic delivery by intravenous, intraperitoneal or subcutaneous injection. In preferred embodiments, the formulations are delivered by subcutaneous injection.

A therapeutically effective dose refers to a dose that produces the therapeutic effects for which it is administered. An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient.

The exact dosage will be determined in light of factors related to the subject requiring treatment, and may be ascertained using standard techniques. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

In general, the anti-myostatin adnectin is administered subcutaneously at about weekly dosages of about 5-200 mg, more preferably about 5-50 mg. In certain embodiments, the anti-myostatin adnectin formulation is administered subcutaneously at weekly dosages of 7.5, 15, 35 and 50 mg. The dose level is based on the patient weight band and the projected suppression of myostatin. In certain embodiments, patients less than 45 kg are dosed with 7.5 mg dose corresponding 70% suppression of myostatin, and patients greater than 45 kg weight are dosed with 15 mg to attain the same level of myostatin suppression. In certain embodiments, patients less than 45 kg weight are dosed with 35 mg dose corresponding to 90% suppression of myostatin, and patients greater than 45 kg weight are dosed with 15 mg to attain the same level (90%) of myostatin suppression

The frequency of dosing will depend upon the pharmacokinetic parameters of the binding agent molecule in the formulation used. Typically, a composition is administered until a dosage is reached that achieves the desired effect. The composition may therefore be administered as a single dose or as multiple doses (at the same or different concentrations/dosages) over time. Further refinement of the appropriate dosage is routinely made. Appropriate dosages may be ascertained through use of appropriate dose-response data. For example, the anti-myostatin Adnectin may be less frequently (e.g., bi-weekly, or monthly). In addition, as is known in the art, adjustments for age as well as the body weight, general health, sex, diet, time of administration, drug interaction, and the severity of the disease may be necessary, and will be ascertainable with routine experimentation by those skilled in the art. The anti-myostatin Adnectin is suitably administered to the patient at one time or over a series of treatments.

### IX. Kits and Articles of Manufacture

The anti-myostatin Adnectin of the invention can be provided in a kit, a packaged combination of reagents in predetermined amounts with instructions for use in the therapeutic or diagnostic methods of the invention.

For example, in one embodiment of the invention, an article of manufacture containing materials useful for the treatment or prevention of the disorders or conditions described above is provided. The article of manufacture comprises a container and a label. Suitable containers include, for example, bottles, vials, syringes, and test tubes. The container may be formed from a variety of materials such as glass, plastic or metals.

The container holds liquid formulations provided herein. The label on, or associated with, the container may indicate directions for storage and/or use. The label may further indicate that the SC formulation is useful or intended for subcutaneous administration. The container holding the formulation may be a multi-use vial, which allows for repeat administrations (e.g. from 2-6 administrations) of, for example, the subcutaneous formulation. Alternatively, the container may be a pre-filled syringe containing, for example, the subcutaneous formulation in unit dosage form.

The article of manufacture may further comprise other materials desirable from a commercial and user standpoint, including diluents, filters, needles, syringes, and package inserts with instructions for use.

### X. Methods of Use

In one aspect, the present invention provides stable formulations of anti-myostatin Adnectins useful for the treatment of myostatin-related disease or disorders, e.g., muscle wasting disorders, muscle atrophy, metabolic disorders, and bone degenerative disorders. Accordingly, in certain embodiments the invention provides methods for attenuating or inhibiting a myostatin-related disease or disorder in a subject comprising administering an effective amount of myostatin-binding polypeptide, i.e., an anti-myostatin Adnectin, to a subject. In some embodiments, the subject is a human. In some embodiments, the anti-myostatin Adnectins are pharmaceutically acceptable to a mammal, in particular a human. A “pharmaceutically acceptable” polypeptide refers to a polypeptide that is administered to an animal without significant adverse medical consequences, such as essentially endotoxin free, or very low endotoxin levels.

The anti-myostatin Adnectins of the present invention can be used to treat muscular, neurological and metabolic disorders associated with muscle wasting and/or muscle atrophy. For example, myostatin overexpression in vivo induces signs and symptoms characteristic of cachexia, and myostatin binding agents can partially resolve the muscle wasting effect of myostatin (Zimmers et al., Science 2002; 296:1486-8). Patients with AIDS also exhibit increased serum levels of myostatin immunoreactive material compared to patients without AIDS or to AIDS patients who do not exhibit weight loss (Gonzalez-Cadavid et al., PNAS 1998; 95:14938-43). It has also been observed that heart-specific elimination of myostatin reduces skeletal muscle atrophy in mice with heart failure, and conversely, specifically overexpressing myostatin in the heart is sufficient to induce muscle wasting (Breitbart et al., AJP-Heart; 2011; 300:H1973-82). In contrast, myostatin knockout mice show increased muscle mass, and an age-dependent decrease in fat accumulation compared to their wild type counterparts (McPherron et al., J. Clin. Invest. 2002; 109:595-601).

Exemplary disorders that can be treated according to the methods of the invention include myopathies and neuropathies, including, for example, motor neuron disease, neuromuscular and neurological disorders.

For example, anti-myostatin Adnectins can be used to treat inherited myopathies and neuromuscular disorders (e.g., muscular dystrophy (Gonzalez-Kadavid et al., PNAS, 1998; 95:14938-43), motor neuron disorders, congenital myopathies, inflammatory myopathies and metabolic myopathies), as well as acquired myopathies (e.g., drug induced myopathy, toxin induced myopathy, infection induced myopathy, paraneoplastic myopathy and other myopathies associated with critical illnesses).

Such disorders include, but are not limited to, Duchenne's muscular dystrophy, progressive muscular dystrophy, Becker's type muscular dystrophy, Dejerine-Landouzy muscular dystrophy, Erb's muscular dystrophy, Emery Dreifuss muscular dystrophy, limb girdle muscular dystrophy, oculopharyngeal muscular dystrophy (OPMD), facioscapulohumeral muscular dystrophy, congenital muscular dystrophy, infantile neuroaxonal muscular dystrophy, myotonic dystrophy (Steinert's disease), distal muscular dystrophy, nemaline myopathy, familial periodic paralysis, nondystrophic myotonia, periodic paralyses, spinal muscular atrophy, spinal muscular atrophy (SMA), amyotrophic lateral sclerosis (ALS), primary lateral sclerosis (PLS), progressive muscular atrophy (PMA), distal myopathy, myotubular/centronuclear myopathy, nemaline myopathy, mini core disease, central core disease, desminopathy, inclusion body myositis, dermatomyositis, polymyositis, mitochondrial myopathy, congenital myasthenic syndrome, myasthenia gravis, post-polio muscle dysfunction, steroid myopathy, alcoholic myopathy, perioperative muscular atrophy and ICU neuromyopathy.

Inherited and acquired neuropathies and radiculopathies which can be treated with anti-myostatin Adnectins include, but are not limited to, rigid spine syndrome, muscle-eye-brain disease, heredity motor and sensory neuropathy, Carcot-Marie-Tooth disease, chronic inflammatory neuropathy, progressive hypertrophic neuropathy, tomaculous neuropathy, lupus, Guillain-Barre syndrome, chronic inflammatory demyelinating polyneuropathy, multiple sclerosis, sarcoidosis, diabetic neuropathy, alcoholic neuropathy, disease related neuropathies (e.g., HIV/AIDS, Lyme disease), toxin related neuropathies (e.g., heavy metal, chemotherapy), compression neuropathies (e.g., tumors, entrapment neuropathy), and neuropathies associated with injury or trauma (e.g., cauda equine syndrome, paraplegia, quadriplegia).

In some embodiments, the anti-myostatin Adnectins of the invention can be used to treat muscular dystrophies (e.g., Duchenne's muscular dystrophy, Becker's type muscular dystrophy), ALS, and sarcopenia.

Additional disorders associated with muscle wasting that can be treated with the anti-myostatin Adnectins of the invention include cachexia, wasting syndrome, sarcopenia, congestive obstructive pulmonary disease, cystic fibrosis (pulmonary cachexia), cardiac disease or failure (cardiac cachexia), cancer, wasting due to AIDS, wasting due to renal failure, renal disease, claudication, cachexia associated with dialysis, uremia, rheumatoid arthritis, muscle injury, surgery, repair of damaged muscle, frailty, disuse atrophy, osteoporosis, osteoarthritis, ligament growth and repair.

The methods of the invention can also be used to increase muscle volume in subjects who suffer from muscle atrophy due to disuse. Disuse atrophy may result from numerous causes including any disorder or state which leads to prolonged immobility or disuse, including, but not limited to prolonged bedrest, being wheelchair bound, limb immobilization, unloading of the diaphragm via mechanical ventilation, solid organ transplant, joint replacement, stroke, CNS damage related weakness, spinal cord injury, recovery from severe burn, sedentary chronic hemodialysis, post-trauma recovery, post-sepsis recovery and exposure to microgravity (Powers et al., Am J Physiol Regul Integr Comp Physiol 2005; 288:R337-44).

In addition, age-related increases in fat to muscle ratios, and age-related muscular atrophy appear to be related to myostatin. For example, the average serum myostatin-immunoreactive protein increased with age in groups of young (19-35 yr old), middle-aged (36-75 yr old), and elderly (76-92 yr old) men and women, while the average muscle mass and fat-free mass declined with age in these groups (Yarasheski et al. J Nutr Aging 6(5):343-8 (2002)). Accordingly, Subjects with muscle atrophy due to aging, and/or subjects who are frail due to, for example, sarcopenia, would also benefit from treatment with the anti-myostatin Adnectins of the invention.

Also contemplated are methods for increasing muscle mass in food animals by administering an effective dosage of the anti-myostatin Adnectins to these animals. Since the mature C-terminal myostatin polypeptide is identical in all species, anti-myostatin Adnectins would be expected to effectively increase muscle mass and reducing fat in any agriculturally important species, for example, but not limited to, cattle, chicken, turkeys, and pigs.

The efficacy of the anti-myostatin Adnectin in the treatment of muscle wasting disorders or muscle atrophy can be determined, for example, by one or more methods for measuring an increase in muscle mass or volume, an increase in the number of muscle cells (hyperplasia), an increase in muscle cell size (hypertrophy) and/or an increase in muscle strength. For example, the muscle volume increasing effects of the anti-myostatin Adnectins of the present invention are demonstrated in the Examples described infra. Methods for determining “increased muscle mass” are well known in the art. For example, muscle content can be measured before and after administration of an anti-myostatin Adnectin of the invention using standard techniques, such as underwater weighing (see, e.g., Bhasin et al. New Eng. J. Med. (1996) 335:1-7) and dual-energy x-ray absorptiometry (see, e.g., Bhasin et al. Mol. Endocrinol. (1998) 83:3155-3162). An increase in muscle size may be evidenced by weight gain of at least about 5-10%, preferably at least about 10-20% or more.

### Metabolic Disorders

The anti-myostatin Adnectins of the present invention, which reduce myostatin activity and/or signaling, are useful for treating metabolic disorders, such as obesity, type II diabetes mellitus, diabetes associated disorders, metabolic syndrome, and hyperglycemia.

Myostatin is involved in the pathogenesis of type II diabetes mellitus. Myostatin is expressed in adipose tissue and myostatin deficient mice exhibit reduced fat accumulation as they age. Moreover, glucose load, fat accumulation, and total body weight are reduced in myostatin lacking agouti lethal yellow and obese (Lep^{ob/ob}) mice (Yen et al., FASEB J. 8:479, 1994; McPherron et al., 2002). As disclosed in US2011/0008375, myostatin antagonists can decrease the fat to muscle ratio in an aged mouse model, preserve skeletal muscle mass and lean body mass, and attenuate kidney hypertrophy in STZ-induced diabetic mice.

As used herein, “obesity” is a condition in which excess body fat has accumulated to such an extent that health may be negatively affected. It is commonly defined as a body mass index (BMI) of 30 kg/m2 or higher which distinguishes it from being overweight as defined by a BMI of 25 kg/m2 or higher (see, e.g., World Health Organization (2000) (PDF). Technical report series 894: Obesity: Preventing and managing the global epidemic. Geneva: World Health Organization). Excessive body weight is associated with various diseases, particularly cardiovascular diseases, diabetes mellitus type II, obstructive sleep apnea, certain types of cancer, and osteoarthritis.

A subject with obesity may be identified, for example, by determining BMI (BMI is calculated by dividing the subject's mass by the square of his or her height), waist circumference and waist-hip ratio (the absolute waist circumference (>102 cm in men and >88 cm in women) and the waist-hip ratio (the circumference of the waist divided by that of the hips of >0.9 for men and >0.85 for women) (see, e.g., Yusuf S, et al., (2004). Lancet 364: 937-52), and/or body fat percentage (total body fat expressed as a percentage of total body weight: men with more than 25% body fat and women with more than 33% body fat are obese; body fat percentage can be estimated from a person's BMI by the following formula: Bodyfat %=(1.2*BMI)+(0.23*age)−5.4−(10.8*gender), where gender is 0 if female and 1 if male). Body fat percentage measurement techniques include, for example, computed tomography (CT scan), magnetic resonance imaging (MRI), and dual energy X-ray absorptiometry (DEXA).

The term “type II diabetes” refers to a chronic, life-long disease that results when the body's insulin does not work effectively. A main component of type II diabetes is “insulin resistance,” wherein the insulin produced by the pancreas cannot connect with fat and muscle cells to allow glucose inside to produce energy, causing hyperglycemia (high blood glucose). To compensate, the pancreas produces more insulin, and cells, sensing this flood of insulin, become even more resistant, resulting in a vicious cycle of high glucose levels and often high insulin levels.

The phrase “disorders associated with diabetes” or “diabetes associated disorders” or “diabetes related disorders,” as used herein, refers to conditions and other diseases which are commonly associated with or related to diabetes. Example of disorders associated with diabetes include, for example, hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis, ulcerative colitis, coronary heart disease, hypertension, angina pectoris, myocardial infarction, stroke, skin and connective tissue disorders, foot ulcerations, metabolic acidosis, arthritis, and osteoporosis.

The efficacy of the anti-myostatin Adnectins in the treatment of metabolic disorders can be determined, for example, by one or more methods of measuring an increase in insulin sensitivity, an increase in glucose uptake by cells from the subject, a decrease in blood glucose levels, and a decrease in body fat.

For example, in subjects having type II diabetes or who are at risk of developing diabetes HbA1c levels can be monitored. The term “hemoglobin 1AC” or “HbA1c” as used herein refers to the product of a non-enzymatic glycation of the hemoglobin B chain. The desired target range of HbA1c levels for people with diabetes can be determined from American Diabetes Association (ADA) guidelines, i.e., the Standards of Medical Care in Diabetes (Diabetes Care 2012; 35(Suppl 1):5511-563). Current HbA1c target levels are generally <7.0% for people with diabetes, and people who do not have diabetes typically have HbA1c values of less than 6%. Accordingly, the efficacy of the anti-myostatin Adnectins of the present invention can be determined by an observed decrease in the HBA1c level in a subject.

The methods of the invention further include administration of an anti-myostatin Adnectin alone, or in combination with other agents that are known in the art for glycemic control (e.g., insulin, GLP1) or for treating art-recognized diabetes-related complications.

### Embodiments

1. A stable pharmaceutical formulation comprising

(i) at least 10 mg/mL of a polypeptide comprising a fibronectin type III tenth (¹⁰Fn3) domain which binds to myostatin;

(ii) a disaccharide at a concentration of at least 5%;

(iii) a histidine buffer at a concentration of between about 20 to about 60 mM; and

(iv) a pharmaceutically acceptable aqueous carrier, wherein the formulation has a pH range of about 6.5 to about 7.8.

2. The formulation of embodiment 1, wherein the protein concentration of the anti-myostatin adnectin in the formulation is between about 10 mg/mL and 200 mg/mL.

3. The formulation of embodiment 1, wherein the protein concentration of the anti-myostatin adnectin in the formulation is between about 10 mg/mL and 150 mg/mL.

4. The formulation of embodiment 1, wherein the protein concentration of the anti-myostatin adnectin is between about 10 mg/mL and 85 mg/mL.

5. The formulation of embodiment 1, wherein the protein concentration of the anti-myostatin adnectin in the formulation is selected from the group consisting of about 10.7 mg/mL, about 21.4 mg/mL, about 50 mg/mL and about 71.4 mg/mL.

6. The formulation of any of the preceding embodiments, wherein the disaccharide is present at weight (w/w) ratio of at least 5:1 protein to sugar.

7. The formulation of any of the preceding embodiments, wherein the protein:disaccharide weight ratio is between about 5:1 to 10:1.

8. The formulation of any of the preceding embodiments, wherein the protein:disaccharide ratio is about 10:1.

9. The formulation of any of the preceding embodiments, wherein the protein:disaccharide ratio is about 6.75:1.

10. The formulation of any of the preceding embodiments, wherein the formulation comprises about 5% to about 30% of the disaccharide

11. The formulation of any of the preceding embodiments, wherein the formulation comprises about 15% to about 25% of the disaccharide.

12. The formulation of any of the preceding embodiments, wherein the formulation comprises about 20% to about 25% of the disaccharide.

13. The formulation of any of the preceding embodiments, wherein the formulation comprises about 18%, 19%, 20%, 21%, 22%, 23%, 24% or about 25% of the disaccharide.

14. The formulation of any of the preceding embodiments, wherein the concentration of the disaccharide is about 150 mM to about 800 mM.

15. The formulation of any of the preceding embodiments, wherein the concentration of the disaccharide in the formulation is about 300 to about 700 mM.

16. The formulation of any of the preceding embodiments, wherein the disaccharide is trehalose.

17. The formulation of embodiment 16, wherein the formulation comprises about 5 to about 30% trehalose.

18. The formulation of embodiment 16 or embodiment 17, wherein the formulation comprises about 15% to about 25% trehalose.

19. The formulation of any one of embodiments 16-18, wherein the formulation comprises about 20% to about 25% trehalose.

20. The formulation of any one of embodiments 16-18, wherein the formulation comprises about 18%, 19%, 20%, 21%, 22%, 23%, 24% or about 25% trehalose.

21. The formulation of embodiment 16, wherein the formulation comprises 22% trehalose.

22. The formulation of embodiment 16, wherein the formulation comprises 23% trehalose.

23. The formulation of any one of the preceding embodiments, wherein the disaccharide is trehalose dihydrate.

24. The formulation of embodiment 23, wherein the concentration of trehalose dihydrate in the formulation is about 150 mM to about 800 mM.

25. The formulation of embodiment 23 or embodiment 24, wherein the concentration of the trehalose dihydrate in the formulation is about 300 to about 700 mM.

26. The formulation of embodiment 24, wherein the concentration of the trehalose dihydrate in the formulation is about 150 mM, about 200 mM, about 250 mM, about 300 mM, about 350 mM, about 400 mM, about 450 mM, about 500 mM, about 550 mM, about 575 mM, about 600, about 625 mM, about 650 mM, about 675 mM or about 700 mM.

27. The formulation of embodiment 24, wherein the concentration of trehalose dihydrate in the formulation is 600 nM.

28. The formulation of any of the preceding embodiments, wherein the histidine is present at a concentration of at least 20 mM.

29. The formulation of any of the preceding embodiments, wherein the histidine is present at a concentration of between about 20 mM and about 40 mM.

30. The formulation of embodiment 29, wherein the histidine is present at a concentration of about 20 mM, about 25 mM, about 30 mM or about 35 mM.

31. The formulation of embodiment 29, wherein the histidine is present at a concentration of about 20 mM.

32. The formulation of embodiment 29, wherein the histidine is present at a concentration of about 25 mM.

33. The formulation of embodiment 29, wherein the histidine is present at a concentration of about 30 mM.

34. The formulation of any of the preceding embodiments, wherein the viscosity of the formulation is from about 5 to 20 cps.

35. The formulation of any of the preceding embodiments, wherein the viscosity of the formulation is from about 5 to 15 cps.

36. The formulation of any of the preceding embodiments, wherein the viscosity of the formulation is 7 to 12 cps.

37. The formulation of embodiment 34, wherein the viscosity of the formulation is less than about 8 cps.

38. The formulation of any of the preceding embodiments, wherein the pH is about 6.6 to 7.6.

39. The formulation of embodiment 38, wherein the pH of the formulation is about 6.8 to 7.4.

40. The formulation of embodiment 38, wherein the pH of the formulation is about 7.0 to 7.3.

41. The formulation of embodiment 38, wherein the pH of the formulation is about 6.9, 7.0, 7.1, 7.2 or 7.3.

42. The formulation of embodiment 38, wherein the pH of the formulation is about 7.1.

43. The formulation of any of the preceding embodiments comprising a surfactant at a concentration of between about 0.01% and 0.5%.

44. The formulation of embodiment 43, wherein concentration of the surfactant is between about 0.02% and about 0.1%.

45. The formulation of embodiment 43 or 44, wherein the surfactant is polysorbate 20 or polysorbate 80.

46. The formulation any one of embodiments 43 to 45, wherein the surfactant is polysorbate 80 at a concentration of 0.02%.

47. The formulation of any of the preceding embodiments comprising a chelator

48. The formulation of embodiment 47, wherein the concentration of the chelator is between about 0.01 mM and about 0.5 mM.

49. The formulation of embodiment 47, wherein the concentration of the chelator is between about 0.05 mM and 0.2 mM.

50. The formulation of any one of embodiments 47 to 49, wherein the chelator is selected from the group consisting of DPTA, EDTA and EGTA.

51. The formulation of any one of embodiments 47 to 50, wherein the chelator is DPTA.

52. The formulating of embodiment 51, wherein the concentration of DPTA is about 0.05 mM.

53. A stable pharmaceutical formulation comprising,
(i) about 10-140 mg/mL of a polypeptide comprising a fibronectin type III tenth (¹⁰Fn3) domain which binds to myostatin;
(ii) about 5-25% trehalose dihydrate;
(iii) about 20-30 mM histidine; and
(iv) a pharmaceutically acceptable aqueous carrier,
   wherein the pH of the formulation is about 6.8 to 7.3.

54. A stable pharmaceutical formulation comprising,
(i) about 10-140 mg/mL of a polypeptide comprising a fibronectin type III tenth (¹⁰Fn3) domain which binds to myostatin;
(ii) about 5-25% trehalose dihydrate;
(iii) about 20-30 mM histidine;
(iv) about 0.02-0.06 mM DTPA;
(v) about 0.01-0.05% polysorbate 80; and
(vi) a pharmaceutically acceptable aqueous carrier,
   wherein the pH of the formulation is about 6.8 to 7.3.

55. A stable pharmaceutical formulation comprising,
(i) about 10-140 mg/mL of a polypeptide comprising a fibronectin type III tenth (¹⁰Fn3) domain which binds to myostatin;
(ii) about 600 mM trehalose dihydrate;
(iii) 25-30 mM histidine; and
(iv) a pharmaceutically acceptable aqueous carrier,
   wherein the pH of the formulation is about 7.0 to 7.3.

56. A stable pharmaceutical formulation comprising
(i) about 10-140 mg/mL of a polypeptide comprising a fibronectin type III tenth (¹⁰Fn3) domain which binds to myostatin;
(ii) about 600 mM trehalose dihydrate;
(iii) 25-30 mM histidine;
(iv) about 0.02-0.06 mM DTPA;
(v) about 0.01-0.05% polysorbate 80; and
(vi) a pharmaceutically acceptable aqueous carrier,
   wherein the pH of the formulation is about 7.0 to 7.3.

57. A stable pharmaceutical formulation comprising,
(i) about 10-75 mg/mL of a polypeptide comprising a fibronectin type III tenth (¹⁰Fn3) domain which binds to myostatin;
(ii) about 5-25% trehalose dihydrate;
(iii) about 20-30 mM histidine; and
(iv) a pharmaceutically acceptable aqueous carrier,
   wherein the pH of the formulation is about 6.8 to 7.3.

58. A stable pharmaceutical formulation comprising,
(i) about 10-75 mg/mL of a polypeptide comprising a fibronectin type III tenth (¹⁰Fn3) domain which binds to myostatin;
(ii) about 5-25% trehalose dihydrate;
(iii) about 20-30 mM histidine;
(iv) about 0.02-0.06 mM DTPA;
(v) about 0.01-0.05% polysorbate 80; and
(vi) a pharmaceutically acceptable aqueous carrier,
   wherein the pH of the formulation is about 6.8 to 7.3.

59. A stable pharmaceutical formulation comprising
(i) about 10-75 mg/mL of a polypeptide comprising a fibronectin type III tenth (¹⁰Fn3) domain which binds to myostatin;
(ii) about 600 mM trehalose dihydrate;
(iii) about 30 mM histidine;
(iv) about 0.05 mM DTPA;
(v) about 0.02% polysorbate 80;
(vi) a pharmaceutically acceptable aqueous carrier,
   wherein the pH of the formulation is about 7.1.

60. The formulation of any one of embodiments 53 to 57, comprising about 10-85 mg/mg mL of the polypeptide.

61. The formulation of any one of embodiments 58 to 60, comprising about 10.7 mg/mL, about 21.4 mg/mL, about 50 mg/mL, or about 71.4 mg/mL of the polypeptide.

62. A unit dosage form comprising about 1.0 mL or less of a formulation comprising,
(i) about 10-75 mg/mL of a polypeptide comprising a fibronectin type III tenth (¹⁰Fn3) domain which binds to myostatin;
(ii) about 5-25% trehalose dihydrate;
(iii) about 20-30 mM histidine;
(iv) about 0.02-0.06 mM DTPA;
(v) about 0.01-0.05% polysorbate 80; and
(vi) a pharmaceutically acceptable aqueous carrier,
   wherein the pH of the formulation is about 6.8 to 7.3.

63. The unit dosage form of embodiment 63, wherein the formulation comprises
(i) about 10-75 mg/mL of a polypeptide comprising a fibronectin type III tenth (¹⁰Fn3) domain which binds to myostatin;
(ii) about 600 mM trehalose dihydrate;
(iii) about 30 mM histidine;
(iv) about 0.05 mM DTPA;
(v) about 0.02% polysorbate 80;
(vi) a pharmaceutically acceptable aqueous carrier,
   wherein the pH of the formulation is about 7.1.

64. The formulation of any one of the preceding embodiments, wherein at least one loop of the BC, DE, and FG loops of the ¹⁰Fn3 domain has 0, 1, 2, or 3 amino acid substitutions relative to the respective BC, DE, and FG loops of SEQ ID NOs: 5, 6 and 7, respectively.

65. The formulation of any of the preceding embodiments, wherein at least one of the BC, DE, and FG loops of the ¹⁰Fn3 domain has 1 amino acid substitution relative to one loop from the BC, DE, or FG loop of SEQ ID NOs: 5, 6 and 7, respectively.

66. The formulation of any of the preceding embodiments, wherein the ¹⁰Fn3 domain has 1 amino acid substitution relative to the respective BC, DE, or FG loop of SEQ ID NOs: 5, 6 and 7, respectively.

67. The formulation of any one of embodiments 1 to 65, wherein the BC, DE, and FG loops of the ¹⁰Fn3 domain comprise the amino acid sequence of SEQ ID NOs: 5, 6 and 7, respectively.

68. The formulation of any one of the preceding embodiments, wherein the ¹⁰Fn3 domain comprises an amino acid sequence at least 90%, 91%, 92%, 93%, 94%, 95%, 96%, 97%, 98% or 99% identical to the non-BC, DE, and FG loop regions of SEQ ID NO: 8, 9 or 10.

69. The formulation of any one of embodiments 1 to 65, wherein the ¹⁰Fn3 domain comprises the amino acid sequence of SEQ ID NO: 8.

70. The formulation of any one of the preceding embodiments, wherein the polypeptide comprises an Fc domain.

71. The formulation of embodiment 71, wherein the polypeptide in the formulation comprises an amino acid sequence at least 90%, 91%, 92%, 93%, 94%, 95%, 96%, 97%, 98% or 99% identical SEQ ID NO: 78.

72. The formulation of embodiment 71, wherein the polypeptide in the formulation comprises the amino acid sequence of SEQ ID NO: 78.

73. The formulation of any of the preceding embodiments, wherein the polypeptide is a dimer.

74. A stable pharmaceutical formulation comprising,
(i) about 10-75 mg/mL of a polypeptide comprising the amino acid of SEQ ID NO: 78;
(ii) about 5-25% trehalose dihydrate;
(iii) about 20-30 mM histidine; and
(iv) a pharmaceutically acceptable aqueous carrier,
   wherein the pH of the formulation is about 6.8 to 7.3.

75. The formulation of embodiment 75, further comprising about 0.02-0.06 mM DTPA.

76. A stable pharmaceutical formulation comprising
(i) about 10-75 mg/mL of a polypeptide comprising the amino acid of SEQ ID NO: 78;
(ii) about 600 mM trehalose dihydrate;
(iii) 25-30 mM histidine;
(iv) about 0.02-0.06 mM DTPA;
(v) about 0.01-0.05% polysorbate 80; and
(vi) a pharmaceutically acceptable aqueous carrier,
   wherein the pH of the formulation is about 7.0 to 7.3.

77. A stable pharmaceutical formulation comprising
(i) about 10-75 mg/mL of a polypeptide comprising the amino acid of SEQ ID NO: 78;
(ii) about 600 mM trehalose dihydrate;
(iii) about 30 mM histidine;
(iv) about 0.05 mM DTPA;
(v) about 0.02% polysorbate 80;
(vi) a pharmaceutically acceptable aqueous carrier,
   wherein the pH of the formulation is about 7.1.

78. The formulation of embodiment 77 or embodiment 78, wherein the formulation comprises about 10.7 mg/mL, about 21.4 mg/mL, about 50 mg/mL or about 71.4 mg/mL of the polypeptide.

79. The formulation of any one of the preceding embodiments formulated for intravenous, intramuscular or subcutaneous injection.

80. The formulation of embodiment 80, embodiment for subcutaneous injection.

81. The formulation of any one of the preceding embodiments provided in unit dosage form at a volume of between about 0.3 mL to 1 mL.

82. The formulation of embodiment 82, wherein the unit dosage form is provided at a volume of about 0.3 mL, 0.4 mL, 0.5 mL, 0.6 mL, 0.7 mL, 0.8 mL, 0.9 mL or 1.0 mL.

83. A method of attenuating or inhibiting a myostatin-related disease or disorder in a subject comprising administering an effective amount of a pharmaceutical formulation of any one of the preceding embodiments.

84. The method of embodiment 84, wherein the myostatin-related disease or disorder is associated with degeneration or wasting of muscle in the subject.

85. The method of embodiment 84, wherein the myostatin-related disease or disorder is a metabolic disorder.

86. The method of embodiment 84, wherein the myostatin-related disease or disorder is selected from the group consisting of Amyotrophic Lateral Sclerosis (ALS), Becker's Muscular Dystrophy (BMD), Spinal Muscular Atrophy and Duchenne Muscular Dystrophy (DMD).

87. The method of embodiment 84, wherein the myostatin-related disease or disorder is sarcopenia or type II diabetes.

88. The method of any one of embodiments 84 to 88, wherein the polypeptide comprising a fibronectin type III tenth (¹⁰Fn3) domain which binds to myostatin is administered at a dosage of about 5 mg to 200 mg.

89. The method of embodiment 89, wherein the polypeptide comprising a fibronectin type III tenth (¹⁰Fn3) domain which binds to myostatin is administered at a dosage of about 7.5, 15, 35, or 50 mg.

90. The method of any one of embodiments 84 to 90, wherein the formulation is administered subcutaneously.

91. The method of any one of embodiments 84 to 91, wherein the formulation is administered at a weekly dosage of about 5 mg to about 200 mg.

92. The method of any one of embodiments 84 to 92, wherein the formulation is administered at a weekly dosage of about 5 mg to about 50 mg.

93. The method of any one of embodiments 84 to 93, wherein the formulation is administered subcutaneously at a weekly dosage of about 7.5 mg, about 15 mg, about 35 mg, or about 50 mg.

94. The method of any one of embodiments 84 to 94, wherein the subject is a pediatric patient less than 21 years of age.

95. The method of embodiment 95, wherein the subject is a pediatric patient between about 6 and 12 years of age.

96. The method of any one of embodiments 84 to 96, wherein the subject is less than about 45 kg and is administered a dosage of about 7.5 mg to about 35 mg.

97. The method of any one of embodiments 84 to 96, wherein the subject is more than about 45 kg and is administered a dosage of about 15 mg to about 50 mg.

### INCORPORATION BY REFERENCE

All documents and references, including patent documents and websites, described herein are individually incorporated by reference to into this document to the same extent as if there were written in this document in full or in part.

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### Example 1

### A. Expression and Purification of Anti-Myostatin Adnectins

Methods for cloning, expressing and purifying insoluble and soluble anti-myostatin adnectins have been previously described (U.S. Pat. Nos. 8,933,199; 8,993,265; 8,853,154; and 9,493,546). Briefly, nucleic acids encoding an anti-myostatin adnectin are cloned into a pET9d vector and expressed in E. coli BL21 DE3 plysS cells. Twenty ml of an inoculum culture (generated from a single plated colony) are used to inoculate 1 liter of LB medium or TB-Overnight Expression Media (auto induction) containing 50 μg/ml Kanamycin and 34 μg/ml chloramphenicol. Cultures in LB medium are incubated at 37° C. until A₆₀₀ 0.6-1.0 and then induced with 1 mM isopropyl-β-thiogalactoside (IPTG) and grown for 4 hours at 30° C. Cultures grown in TB-Overnight Expression Media are incubated at 37° C. for 5 hours, at which time the temperature was lowered to 18° C. and grown for 19 hours. Cultures are harvested by centrifugation for 30 minutes at 10,000 g at 4° C. Cell pellets were frozen at −80° C. After thawing, the cell pellet are resuspended in 25 ml of lysis buffer (20 mM NaH₂PO₄, 0.5 M NaCl, 1× Complete™ Protease Inhibitor Cocktail-EDTA free (Roche), pH 7.4) using an Ultra-turrax homogenizer (IKA works) on ice. Cell lysis is achieved by high pressure homogenization (≥18,000 psi) using a Model M-110S Microfluidizer (Microfluidics).

For insoluble anti-myostatin adnectins, the insoluble fraction is separated by centrifugation for 30 minutes at ≥23,300 g at 4° C. The insoluble pellet recovered from centrifugation of the lysate is washed with 20 mM sodium phosphate/500 mM NaCl, pH7.4. The pellet was resolubilized in 6 M guanidine hydrochloride in 20 mM sodium phosphate/500 mM NaCl pH 7.4 with sonication, followed by incubation at 37° degrees for 1-2 hours. The resolubilized pellet is filtered with a 0.45 μm filter and loaded onto a Histrap column equilibrated with the 20 mM sodium phosphate/500 mM NaCl/6 M guanidine pH7.4 buffer. After loading, the column is washed for an additional 25 column volumes with the same buffer. Bound protein was eluted with 50 mM imidazole in 20 mM sodium phosphate/500 mM NaCl/6 M guanidine-HCl, pH 7.4. The purified protein is refolded by dialysis against 50 mM sodium acetate/150 mM NaCl, pH 4.5 or PBS, pH 7.2.

For soluble anti-myostatin adnectins, the supernatant is clarified using a 0.45 μm filter. The clarified lysate is loaded onto a Histrap column (GE) pre-equilibrated with 20 mM sodium phosphate/500 mM NaCl, pH 7.4. The column is then washed with 25 column volumes of the same buffer, followed by 20 column volumes of 20 mM sodium phosphate/500 mM NaCl/25 mM imidazole, pH 7.4 and then 35 column volumes of 20 mM sodium phosphate/500 mM NaCl/40 mM imidazole, pH 7.4. Protein is eluted with 15 column volumes of 20 mM sodium phosphate/500 mM NaCl/500 mM imidazole, pH 7.4, fractions were pooled based on absorbance at A₂₈₀, and dialyzed against 1×PBS or 50 mM Tris, 150 mM NaCl, pH 8.5 or 50 mM NaOAc, 150 mM NaCl, pH 4.5. Precipitates are removed by filtering with a 0.22 μm filter.

### B. Expression and Purification of Fc-Formatted Anti-Myostatin Adnectins

For DNA generation, nucleic acid encoding the selected anti-myostatin adnectins were cloned into a pDV-16 plasmid from which E. coli Top10 cells were transformed. pDV-16 is a modified version of pTT5 (Yves Durocher, NRC Canada), wherein the human IgG1-Fc coding sequence has been introduced, preceded by signal sequence, and restriction sites were included to allow insertion of Adnectin coding sequences at either terminus of the Fc. Transformed cells were expanded by inoculating 1 L of Luria broth containing 100 μg/ml Ampicillin and incubating in a rotating incubator at 225 rpm for 18 hours at 37° C. Bacterial pellets were harvested by centrifugation at >10000 g for 30 minutes at 4° C. Purified plasmid DNA was isolated using a QIAGEN Plasmid Plus Mega Kit (QIAGEN) as described in the manufacturer's protocol. Purified DNA was quantified using absorbance at 260 nm and frozen at −80° C. prior to use.

HEK 293-EBNA1 (clone 6E) (Yves Durocher, NRC Canada) cells were expanded to 2×10⁶ cells/ml in 2 L of F17 media in a 10 L GE Healthcare Wave bag at 37° C., 5% CO₂, and mixed by rocking at an 8 degree angle at 18 rpm.

DNA was prepared for transfection as follows: F17 media was warmed to 37° C. DNA and a PEI transfection reagent were thawed in a sterile biosafety hood. DNA (2.25 mg) was added to 100 ml of warmed F17 media in a sterile polypropylene culture flask and gently mixed by swirling. In a separate flask, 6.75 mg of PEI (1 mg/ml) was combined with 100 ml of pre-warmed F17 media and gently mixed by swirling. The flasks were allowed to rest for 5 minutes prior to combining the contents by adding the PEI solution to the flask containing the DNA and gently mixing by swirling.

The contents of the flask containing the DNA:PEI mixture were added to the wave bag containing the HEK 293-6E cells after incubating at room temperature for 15 minutes in the biosafety hood. The bag containing the transfected HEK 293-6E cells was incubated for twenty four hours at 37° C., 5% CO₂, and mixed by rocking at an 8 degree angle at 18 RPM. After 24 hours, 100 ml of sterile filtered 20% Tryptone N1 (Organotechnie, Canada) dissolved in F17 media was aseptically added to the culture. The cells and media were harvested after an additional 72 hours of incubation as described above. Alternatively, transient HEK expression in shake flasks (0.5 L media in a 2 L flask) can be performed with a DNA:PEI ratio of 1:2. Cells were separated from the conditioned media by centrifugation at 6000 g for 30 minutes at 4° C. The conditioned media was retained, filtered through a 0.2 μM filter, and stored at 4° C.

The conditioned media was applied to a 10 ml chromatography column containing GE MabSelect Sure resin pre-equilibrated in PBS at a rate of 5 ml/minute. After loading the filtered conditioned media, the column was washed with at least 100 ml of PBS at room temperature. The purified product was eluted from the column with the application of 100 mM Glycine/100 mM NaCl, pH 3.0. Fractions were neutralized in pH either by collecting into tubes containing ⅙ volume of 1M Tris pH 8, or by pooling according to A280 absorbance followed by addition of 1M Tris pH 8 to 100 mM. If the content of high molecular weight species is greater than 5% after Protein A elution, then the sample is further purified by a Superdex 200 (26/60) column (GE Healthcare) in PBS. The SEC fractions containing monomers are pooled and concentrated. The resulting protein A or SEC pool was exhaustively dialyzed against PBS at 4° C., and sterile filtered using a 0.22 μm cutoff filter prior to freezing at −80° C.

### C. Bulk Manufacturing: Mammalian Expression and Primary Recovery: UCOE CHO System

A mammalian Research Cell Bank (RCB) was created by transfecting anti-myostatin Adnectin-Fc fusions cloned into the pUCOE vector containing the Ubiquitous Chromatin Opening Element (UCOE) [Modified UCOE vector from Millipore] in CHO-S cells. An RCB was established by expanding cells in selection media (0.04% (v/v) L-Glutamine (Invitrogen) and 0.01% (v/v) HT Supplement (Invitrogen) in CD CHO medium (Invitrogen)) containing 12.5 μg/mL puromycin. Low passage number cells were aseptically isolated via centrifugation, resuspended in banking media (0.04% (v/v) L-Glutamine (Invitrogen), 0.01% (v/v) HT Supplement (Invitrogen) and 7.5% (v/v) DMSO in CD CHO medium (Invitrogen)) to a final concentration of 1×10⁷ cells/mL. These cells were initially frozen in a 70% isopropyl alcohol bath at −80° C. overnight and then transferred to liquid nitrogen for long term storage the following day.

Cell culture was initiated by thawing a single RCB vial into 25 mL of selection media containing 12.5 μg/mL puromycin and expanding the culture in the same media. Cells were allowed to reach a concentration between 1-2×10⁶ cells/mL before being split back to 0.2×10⁶ cells/mL. Cells were generally maintained between 2-4 weeks prior to seeding a bioreactor. The expansion culture was passaged a final time and allowed to grow to the point where a 15 L bioreactor containing 8 L of production media (Invitrogen CD CHO media containing 0.01% (v/v) HT Supplement (Invitrogen), 0.04% (v/v) Glutamax (Gibco), and 0.005% (v/v) Pluronic F-68 (Gibco)) could be seeded at a final density of 0.2×10⁶ cells/mL. The bioreactor culture was monitored daily for VCD (Viable Cell Density), percent Viability, pH, and glucose concentration. The bioreactor culture was fed on days 3 and 6 with a 10% total volume bolus addition of Feed Media. The culture was harvested between Day 7 and Day 9 with a percent viability >70%. During culture, the bioreactor culture was controlled at a pH of 7.1, temperature of 37° C., % DO2 of 40%, and a constant RPM of 100.

On the day of harvest, the bioreactor cultures were directly passed through a 6.0/3.0 μm depth filter followed by a sterile 0.8/0.2 μm filtration into a sterile bag. Clarified sterile culture was stored overnight at 2-8° C. The clarified culture was then concentrated via flatsheet TFF using a 30,000 kDa membrane. The approximate concentration was 6×, depending on harvest titer. Concentrated supernatant was then sterile filtered into PETG bottles and either processed directly or stored at −80° C.

### D. Anti-Myostatin-Adnectin-Fc Fusion Purification

Harvested culture supernatant (neat or concentrated) is loaded onto a MabSelect Protein A column previously equilibrated with PBS. Column is washed with 5CV of 50 mM Tris pH8.0, 1M Urea, 10% PG. Adnectin-Fc fusion is eluted with 100 mM Glycine pH 3.3, collecting the peak into a container which is previously charged with 1CV of 200 mM Sodium Acetate pH 4.5. Peak elution is based on absorbance at A280.

The Protein A elution is diluted to pH 3.0 with the addition of 2 M Citric Acid and left at room temperature for 1 hour, for viral inactivation. Sample is then diluted with 200 mM Sodium Phosphate Tribasic until pH 4.5 is reached. If necessary, the solution is further diluted with water to lower conductivity below 10 ms/cm.

The diluted Protein A elution is passed over a Tosoh Q 600C AR (Tosoh Bioscience), previously conditioned with 50 mM Sodium Acetate pH 4.5, in a negative capture mode. The flowthrough peak is collected, based on absorbance at A280. The column is washed with 50 mM Sodium Acetate and stripped with 0.2N NaOH.

The Q 600C AR flowthrough is formulated using tangential flow filtration utilizing a 30 kD MWCO hollow fiber membrane (GE), with very gentle mixing of the retentate. The adnectin-Fc fusion is diafiltered into Histidine (20-30 mM) and disaccharide (300 to 600 mM) pH 7.1-7.8 for 5 to 8 diavolumes, and then concentrated to a target protein concentration. Bulk volumes of the purified Adnectin-Fc fusion are stored at −60° C. in 12 L FFtp bags at a concentration of 85-140 mg/mL. The purified Adnectin-Fc fusion protein was then thawed and diluted to the desired protein concentration for analysis.

### Example 2

In this study, % HMW formation and % LMW formation was studied for anti-myostatin adnectin in 25 mM Histidine buffer, pH 6.9 containing either Sucrose or Trehalose sugars.

TABLE 1
Anti-myostatin adnectin drug product (DP) properties

| | |
|---|---|
| Description | Dimer of anti-human myostatin antagonist adnectin |
| | which has been formatted with a human IgG1 wild type |
| | Fc (monomer -SEQ ID NO: 78) |
| Molecular | Approximately 75,587 Daltons |
| Weight | |
| Appearance | Clear to slightly opalescent, colorless to pale yellow |
| | solution, essentially free of particulate matter |
| Packaging | Schott 5-cc type I borosilicate glass vial (SAP# |
| | 1334424) with a 20-mm Daikyo butyl D-21-7-S |
| | reformulated B2-40/Flurotec stopper (SAP# 1292587 |
| | ready-to-use or 1239067 ready-to-sterilize) and 20-mm |
| | flipoff seal (SAP# 1187393) |

TABLE 2
Materials

| | | | CSO | | |
|---|---|---|---|---|---|
| Name | Role | Manufacturer | SAP# | Catalog # | Lot # |
| Drug | Active | BMS | — | | PR14018 |
| Substance | Ingredient | | | | |
| L-Histidine | Buffer | J T Baker | — | 2080-06 | J42609 |
| Sucrose | Excipient | BMS | 1020056 | | 2J73171 |
| Trehalose | Excipient | Ferro | — | T-104-1- | 33265A |
| Dihydrate | | (Pfanstiehl) | | MC | |
| Schott 3 cc | Container/ | BMS | 1315365 | | |
| vials | Packaging | | | | |
| Serum | Container/ | Schott | — | | |
| Stoppers | Packaging | | | | |
| Amicon | Disposable | Millipore | — | UFC903096 | |
| Concentrators | | | | | |
| D-tube dialyzers | Disposable | Calbiochem | — | 71746-4 | |
| Stericup | Disposable | EMD | — | | |
| filter units | | Millipore | | | |

TABLE 3
Formulations

| | Protein conc. | Histidine* | Sucrose | Trehalose |
|---|---|---|---|---|
| Formulation # | (mg/mL) | (mM) | (%) | (%) |
| 10% Sucrose | 135 | 25 | 10 | |
| 20% Sucrose | 135 | 25 | 20 | |
| 10% Trehalose | 135 | 25 | | 10 |
| 20% Trehalose | 135 | 25 | | 20 |

*pH adjusted to 7.3

### A. Sample Preparation

For the anti-myostatin adnectin at 135 mg/mL protein concentration, a pH shift of −0.4 pH units was seen upon dialysis in histidine buffer. To circumvent this issue and to be able to assess stability of the formulations at near neutral pH, the buffer pH was adjusted to pH 7.3 so the resultant protein solution would be at pH 6.9.

TABLE 4
Buffer preparation
Preparing 2 liters of Buffer

| | | | | | Trehalose | |
|---|---|---|---|---|---|---|
| | Histidine | | Sucrose | | Dihydrate | |
| | MW | Histidine | MW | Sucrose | MW | Trehalose |
| Formulation# | (g/mol) | (g) | (g/mol) | (g) | (g/mol) | (g) |
| 10% Sucrose | 155.2 | 7.8 | 342.3 | 200 | — | — |
| 20% Sucrose | | 7.8 | | 400 | — | — |
| 10% Trehalose | | 7.8 | — | — | 378.33 | 200 |
| 20% Trehalose | | 7.8 | — | — | 378.33 | 400 |

Appropriate amounts of solids were dissolved in 1600 L Milli-Q water (according to table). The pH was adjusted to pH 7.3 using 6N HCl, and the final volume was adjusted to 2 L. The solutions were filtered through 0.22 μm filter, and the final pH obtained for the buffers is shown in Table 5.

TABLE 5
pH for formulation pH before and after adjustment using 6N HCl

| Formulation | Initial pH | Final pH |
|---|---|---|
| 10% Sucrose | 7.8 | 7.3 |
| 20% Sucrose | 7.8 | 7.3 |
| 10% Trehalose | 7.8 | 7.3 |
| 20% Trehalose | 7.7 | 7.3 |

Forty (40) mL of the anti-myostatin adnectin DP at 50 mg/mL was dialyzed against the different formulation buffers for 5 cycles including one cycle at 5° C.

TABLE 6
Concentration adjustment after dialysis and concentration

| Formulation | Protein Concentration (After Dialysis) mg/mL |
|---|---|
| 10% Sucrose | 165.7 |
| 20% Sucrose | 156.1 |
| 10% Trehalose | 169.4 |
| 20% Trehalose | 154.6 |

The dialyzed solution was concentrated to 130-140 mg/mL for each condition, and the samples were stored at 5° C., 25° C. and 35° C.

### B. Results

### Formulation Characteristics at Time Zero (T0)

At time zero (T0), the appearance of each of the formulations was evaluated by visually inspecting undiluted samples for particle formation. None of the samples showed the presence of visual particulates.

Undiluted samples of each of the formulations were equilibrated at room temperature, and the pH was measured using a Thermo pH meter with Thermo Ross pHerpect pH probe or Orion 3 Star (Manufacturer: Thermo Electron Corporation) according to the manufacturer's instructions for instrument calibration and sample measurement (calibration slope 96.5%). As shown in Table 7, in spite of the pH adjustment of the formulation buffers, the final pH of the samples was still between 6.4-6.6 at T0 indicating that, surprisingly, the protein plays an important buffering role in the formulation.

TABLE 7
pH measurements at T0

| Condition | pH |
|---|---|
| 10% Sucrose | 6.56 |
| 20% Sucrose | 6.52 |
| 10% Trehalose | 6.49 |
| 20% Trehalose | 6.48 |

Samples of the undiluted formulations were equilibrated at room temperature, and protein concentration measurements were performed using SoloVPE following the manufacturer's instructions. All concentrations were within 5% of the target concentration of 135 mg/mL. The results are depicted in Table 8.

TABLE 8
Concentration measurements at T0

| Conditions | Average (mg/ml) |
|---|---|
| 10% Sucrose | 136.6 |
| 20% Sucrose | 137.0 |
| 10% Trehalose | 135.0 |
| 20% Trehalose | 139.0 |

Osmolalilty measurements were performed on undiluted samples of each formulation using vapro based osmometer (Manufacturer: Wescor; Model #5520) according to the manufacturer's instructions (sample volume 10 μL). Additional samples were diluted in their respective buffers to a final concentration of 50 mg/mL and the osmolality measurements were repeated. The results are shown in Table 9.

TABLE 9
Osmolality measurements for formulation
buffer and protein samples

| | Osmolality (mmol/kg) | | |
|---|---|---|---|
| | | Formulation | Formulation |
| | Buffer | (135 mg/mL) | (50 mg/mL) |
| 10% Sucrose | 350 | 401 | 416 |
| 20% Sucrose | 733 | 805 | 797 |
| 10% Trehalose | 322 | 349 | 345 |
| 20% Trehalose | 651 | 735 | 698 |

Trehalose showed the lowest osmolality as compared to same concentration level of sucrose. Relative to the formulation buffer itself, the samples at 50 and 135 mg/mL showed slightly increased osmolality indicating the effect of protein on the osmolality. However, the difference between 50 and 135 mg/mL samples was minimal suggesting that protein concentration has minimal effect on osmolality of the formulation.

Viscosity measurements were performed using m-VROC Viscometer (Manufacturer: Rheosense) (sample volume was 500 μL) at different flowrates (from 30 to 300 μL/min) at 25° C. Formulations with 10% saccharide concentrations had lower viscosity than their 20% counterparts. For comparison of saccharides at each concentration, trehalose showed to possess lower viscosity than sucrose. The results are shown in Table 10.

TABLE 10
Viscosity Measurement for Protein
Samples at 135 mg/mL at 25° C.

| Formulation | Viscosity (mPa-s) |
|---|---|
| 10% Sucrose | 7.6 |
| 20% Sucrose | 12.6 |
| 10% Trehalose | 7.4 |
| 20% Trehalose | 11.1 |

### Formulation Stability Over Time

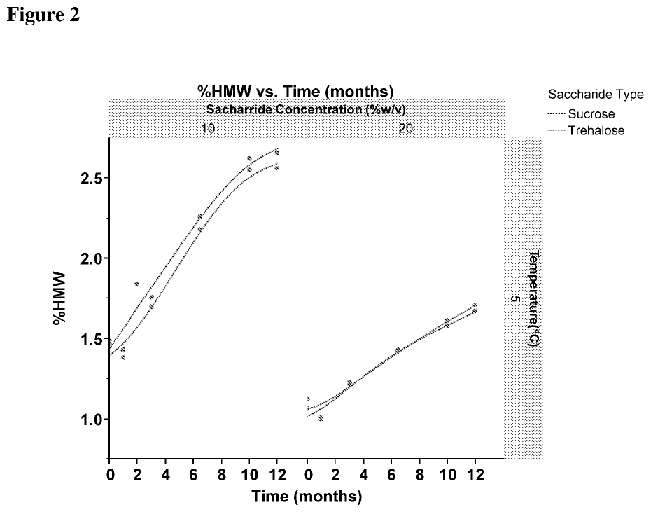

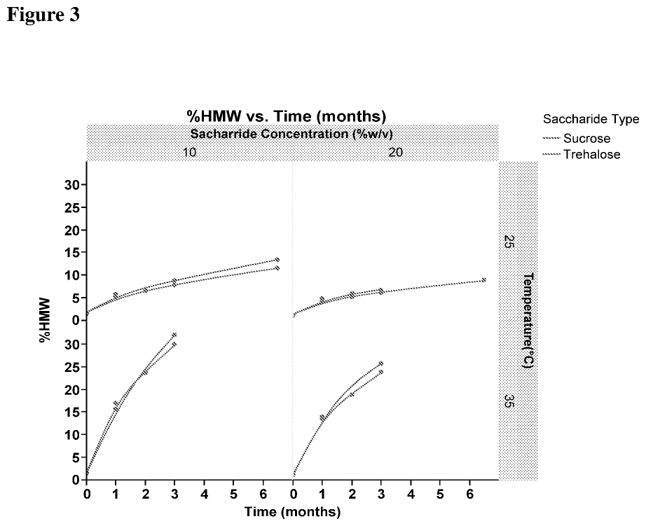

To measure the formation of aggregates over time, SE-HPLC was performed at various time points on samples that were kept at 5° C., 25° C. and 35° C. Samples containing 20% saccharides showed a lower % HMW than their 10% counterparts, indicating that higher saccharide content in formulation was beneficial to the stability of the anti-myostatin adnectin DP. Surprisingly, formulations containing trehalose demonstrated even higher stability of the anti-myostatin adnectin DP compared to sucrose, particularly at higher temperatures (25° C. and 35° C.). (FIGS. 2 and 3).

### The Effect of pH

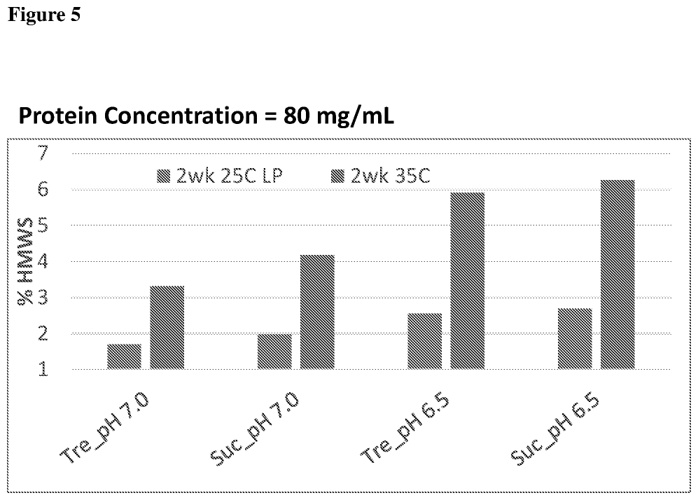

The % HMWS of formulations containing 80 mg/mL of the anti-myostatin adnectin in the presence of either sucrose or trehalose at pH 6.5 and 7.0 were examined after storage for 2 weeks at 25° C. and 35° C. The data shown in FIG. 5 indicates that formulations at pH 7.0 are more stable at both temperatures.

In addition, it was observed that Histidine buffer surprisingly demonstrated better stabilization properties (less HMWS) at pH 7.0 than previous formulations containing phosphate buffer (data not shown).

### The Effect of the Addition of Chelating Agent

The effect of the addition chelating agents to the formulation in the presence and absence of Fe²⁺ was also investigated. The data demonstrated that the addition of 50 μM DPTA further stabilized the formulation was also examined with reduction in the % HMW of >20% in the presence and absence of Fe at for 1 month at either room temperature (light exposed) and 35° C. (e.g., 2.8% v. 3.6%; 2.8% v. 3.3%).

### Example 3

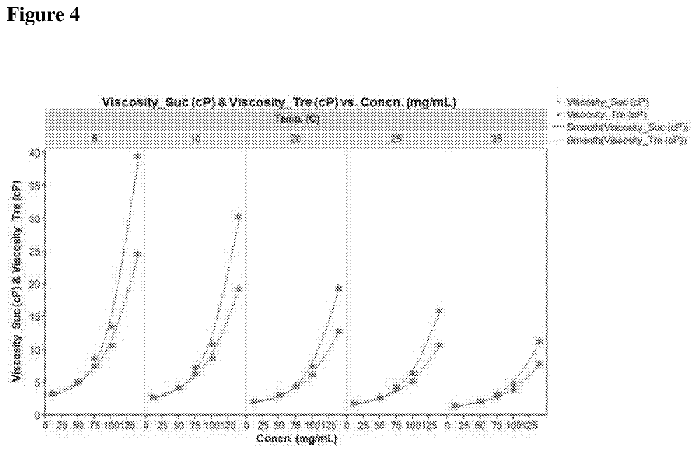

In this example, the viscosity of certain formulations containing the anti-human myostatin antagonist adnectin-Fc fusion dimer from Example 2 was measured in centipoises as indicated in the following Table 11 and FIG. 4 (The trehalose (tre) and sucrose (suc) are indicated in the legend). The measurements were done as function of protein concentration and temperature of solution. The data indicate that formulations containing high disaccharide concentrations (550 mM) generally exhibit viscosities suitable for subcutaneous use at a wide range of anti-myostatin adnectin concentrations. The data also indicate lower viscosities of the solutions containing trehalose as compared to the viscosities of the solutions, under the same temperature and protein concentration, containing sucrose.

TABLE 11

| | | Viscosity (cP) | |
|---|---|---|---|
| Protein | | 20 mM Histidine, | 20 mM Histidine, |
| Conc. | Temperature | 550 mM Sucrose, | 550 mM Trehalose, |
| (mg/mL) | (° C.) | pH 7.0 | pH 7.0 |
| 10 | 5 | 3.3 | 3.3 |
| 10 | 10 | 2.8 | 2.8 |
| 10 | 20 | 2.1 | 2.1 |
| 10 | 25 | 1.8 | 1.8 |
| 10 | 35 | 1.4 | 1.4 |
| 50 | 5 | 5.0 | 5.2 |
| 50 | 10 | 4.2 | 4.3 |
| 50 | 20 | 3.1 | 3.1 |
| 50 | 25 | 2.7 | 2.7 |
| 50 | 35 | 2.1 | 2.1 |
| 75 | 5 | 8.7 | 7.5 |
| 75 | 10 | 7.2 | 6.3 |
| 75 | 20 | 4.5 | 4.5 |
| 75 | 25 | 4.3 | 3.9 |
| 75 | 35 | 3.3 | 2.9 |
| 100 | 5 | 13.5 | 10.6 |
| 100 | 10 | 10.9 | 8.7 |
| 100 | 20 | 7.5 | 6.1 |
| 100 | 25 | 6.4 | 5.2 |
| 100 | 35 | 4.7 | 3.9 |
| 140 | 5 | 39.5 | 24.6 |
| 140 | 10 | 30.3 | 19.3 |
| 140 | 20 | 19.4 | 12.8 |
| 140 | 25 | 16.0 | 10.7 |
| 140 | 35 | 11.3 | 7.8 |

### Example 4

In this example, the anti-myostatin-Fc fusion dimer used in Example 2 was formulated at various protein concentrations in 30 mM Histidine, 600 mM Trehalose, 0.05 mM DPTA, 0.02% PS80 at pH 7.1.

Unit dosages of were prepared in 1 mL syringes at a volume of 0.7 mL at protein concentrations of 10.7 mg/mL, 21.4 mg/mL, 50 mg/mL and 71.4 mg/mL (total drug product 7.5 mg, 15 mg, 35 mg and 50 mg, respectively). The syringes were stored horizontally under various storage conditions and analyzed by SE-HPLC at 2 weeks and/or 1 month. The data are provided in Tables 12-15 (H=Horizontal; RH=Relative Humidity; RL=Room Light; E=Exposed; P=Protected).

TABLE 12
Stability Data for 7.5 mg/Syringe, 1 mL Type 1 Glass Syringe

| | | | SE-HPLC | | |
|---|---|---|---|---|---|
| | | | Monomer | HMW | LMW |
| Condition | Time | | Area % | Area % | Area % |
| Initial | T₀ | | 99.7 | 0.3 | <0.1 |
| 5° C. H | 1 | Month | 99.8 | 0.2 | ND |
| −20° C. H | 1 | Month | 99.8 | 0.2 | ND |
| 25° C./60% RH/H | 1 | Month | 99.8 | 0.2 | ND |
| 40° C./75% RH/H | 2 | Weeks | 99.8 | 0.2 | ND |
| | 1 | Month | 99.7 | 0.3 | ND |
| 25° C./60% RH/RL/E/H | 1 | Weeks | 99.8 | 0.2 | ND |
| | 2 | Weeks | 99.6 | 0.4 | ND |
| | 1 | Month | 99.2 | 0.7 | 0.1 |
| 25° C./60% RH/RL/P/H | 1 | Weeks | 99.8 | 0.2 | ND |
| | 2 | Weeks | 99.8 | 0.2 | ND |
| | 1 | Month | 99.7 | 0.2 | 0.1 |

TABLE 13
Stability Data for 15.0 mg/Syringe, 1-mL Type 1 Glass Syringe

| | | | SE-HPLC | | |
|---|---|---|---|---|---|
| | | | Monomer | HMW | LMW |
| Condition | Time | | Area % | Area % | Area % |
| Initial | T₀ | | 99.7 | 0.3 | <0.1 |
| 5° C. H | 1 | Month | 99.7 | 0.3 | ND |
| −20° C. H | 1 | Month | 99.7 | 0.3 | ND |
| 25° C./60% RH/H | 1 | Month | 99.7 | 0.3 | ND |
| 40° C./75% RH/H | 2 | Weeks | 99.5 | 0.5 | ND |
| | 1 | Month | 99.4 | 0.6 | ND |
| 25° C./60% RH/RL/E/H | 1 | Weeks | 99.7 | 0.3 | ND |
| | 2 | Weeks | 99.4 | 0.6 | ND |
| | 1 | Month | 98.9 | 1.0 | 0.1 |
| 25° C./60% RH/RL/P/H | 1 | Weeks | 99.7 | 0.3 | ND |
| | 2 | Weeks | 99.7 | 0.3 | ND |
| | 1 | Month | 99.6 | 0.3 | 0.1 |

TABLE 14
Stability Data for 35.0 mg/Syringe, 1-mL Type 1 Glass Syringe

| | | | SE-HPLC | | |
|---|---|---|---|---|---|
| | | | Monomer | HMW | LMW |
| Condition | Time | | Area % | Area % | Area % |
| Initial | T₀ | | 99.5 | 0.5 | <0.1 |
| 5° C.H | 1 | Month | 99.5 | 0.5 | ND |
| −20° C. H | 1 | Month | 99.4 | 0.6 | ND |
| 25° C./60% RH/H | 1 | Month | 99.2 | 0.8 | ND |
| 40° C./75% RH/H | 2 | Weeks | 98.2 | 1.8 | ND |
| | 1 | Month | 97.8 | 2.2 | ND |
| 25° C./60% RH/RL/E/H | 1 | Weeks | 99.0 | 1.0 | ND |
| | 2 | Weeks | 98.5 | 1.5 | ND |
| | 1 | Month | 97.8 | 2.1 | 0.1 |
| 25° C./60% RH/RL/P H | 1 | Weeks | 99.4 | 0.6 | ND |
| | 2 | Weeks | 99.4 | 0.6 | ND |
| | 1 | Month | 99.1 | 0.8 | 0.0 |

TABLE 15
Stability Data for 50.0 mg/Syringe, 1-mL Type 1 Glass Syringe

| | | | SE-HPLC | | |
|---|---|---|---|---|---|
| | | | Monomer | HMW | LMW |
| Condition | Time | | Area % | Area % | Area % |
| Initial | T₀ | | 99.3 | 0.6 | <0.1 |
| 5° C.H | 1 | Month | 99.4 | 0.6 | ND |
| -20° C. H | 1 | Month | 99.3 | 0.7 | ND |
| 25° C./60% RH/H | 1 | Month | 98.8 | 1.2 | ND |
| 40° C./75% RH/H | 2 | Weeks | 96.8 | 3.2 | ND |
| | 1 | Month | 96.3 | 3.7 | ND |
| 25° C./60% RH/RL/E/H | 1 | Weeks | 98.6 | 1.4 | ND |
| | 2 | Weeks | 97.9 | 2.1 | ND |
| | 1 | Month | 96.6 | 3.3 | 0.1 |
| 25° C./60% RH/RL/P H | 1 | Weeks | 99.7 | 0.3 | <0.1 |
| | 2 | Weeks | 99.0 | 1.0 | ND |
| | 1 | Month | 98.8 | 1.2 | 0.1 |

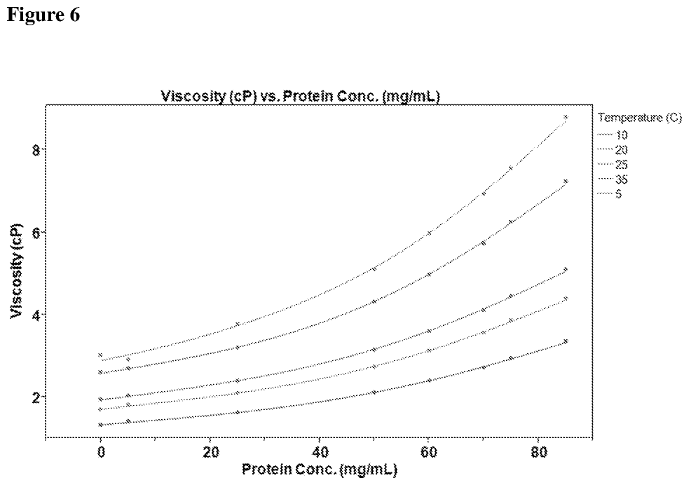

The viscosity of this formulation was also examined. The data shown in FIG. 6 demonstrates that the viscosity of the formulation remained below 8 cPs at all temperatures and protein concentrations.

Based on the favorable viscosity data, the dynamic forces of the formulation in unit dosage form were measured The extrusion (gliding) forces of the 5 mg/mL, 50 mg/mL and 75 mg/mL unit dosages (0.7/mL volume in 1.0 mL syringe; 27 G needle) at 5° C. at a speed of 120 mm/min were between 2.528 and 2.704 N; and between 5.696 and 6.123N at a speed of 450 mm/min. The hydrodynamic forces of these unit dosages at 5° C. at a speed of 120 mm/min were between 0.922 N and 1.098 N, and between 3.042 and 3.509 N at a speed of 450 mm/min between.

### CONCLUSIONS

These results demonstrate that formulations with concentrations of disaccharides above 10% contribute significantly to the stability of the anti-myostatin adnectin molecule while still maintaining an osmolality and viscosity which allow production of unit dosage forms in small volumes suitable for rapid, subcutaneous administration.

The results further show that, surprisingly, the inherent buffering capacity of the anti-myostatin adnectin allows formulation in a histidine buffer at a pH of 6.9-7.3, significantly away from the pKa of the buffer, to produce a formulation which is stable at physiological pH.

These advantageous features provide formulations which are stable for significant periods at higher temperatures, e.g., above 25° C., above 30° C., above 35° C., or up to 40° C., allowing storage and administration outside of a medical facility. The property is particularly advantageous in that it allows patients or their caregivers to administer the drug at home without the need to travel to a medical facility.

Summary of Amino Acid and Nucleic Acid Sequences

Human prepromyostatin:
MQKLQLCVYIYLFMLIVAGPVDLNENSEQKENVEKEGLCNACTWRQNTKSSRIEAIKIQILSKL
RLETAPNISKDVIRQLLPKAPPLRELIDQYDVQRDDSSDGSLEDDDYHATTETIITMPTESDFL
MQVDGKPKCCFFKFSSKIQYNKVVKAQLWIYLRPVETPTTVFVQILRLIKPMKDGTRYTGIRSL
KLDMNPGTGIWQSIDVKTVLQNWLKQPESNLGIEIKALDENGHDLAVTFPGPGEDGLNPFLEVK
VTDTPKRSRRDFGLDCDEHSTESRCCRYPLTVDFEAFGWDWIIAPKRYKANYCSGECEFVFLQK
YPHTHLVHQANPRGSAGPCCTPTKMSPINMLYFNGKEQIIYGKIPAMVVDRCGCS (SEQ ID
NO: 1)
Human pro-myostatin :
NENSEQKENVEKEGLCNACTWRQNTKSSRIEAIKIQILSKLRLETAPNISKDVIRQLLPKAPPL
RELIDQYDVQRDDSSDGSLEDDDYHATTETIITMPTESDFLMQVDGKPKCCFFKFSSKIQYNKV
VKAQLWIYLRPVETPTTVFVQILRLIKPMKDGTRYTGIRSLKLDMNPGTGIWQSIDVKTVLQNW
LKQPESNLGIEIKALDENGHDLAVTFPGPGEDGLNPFLEVKVTDTPKRSRRDFGLDCDEHSTES
RCCRYPLTVDFEAFGWDWIIAPKRYKANYCSGECEFVFLQKYPHTHLVHQANPRGSAGPCCTPT
KMSPINMLYFNGKEQIIYGKIPAMVVDRCGCS (SEQ ID NO: 2)
Mature myostatin:
DFGLDCDEHSTESRCCRYPLTVDFEAFGWDWIIAPKRYKANYCSGECEFVFLQKYPHTHLVHQA
NPRGSAGPCCTPTKMSPINMLYFNGKEQIIYGKIPAMVVDRCGCS (SEQ ID NO: 3)
Wild-type human fibronectin type III domain (¹⁰Fn3):
VSDVPRDLEVVAATPTSLLISWDAPAVTVRYYRITYGETGGNSPVQEFTVPGSKSTATISGLKP
GVDYTITVYAVTGRGDSPASSKPISINYRT (SEQ ID NO: 4) (BC, DE, and FG
loops are underlined)
Anti-myostatin adnectin BC loop:
SWSLPHQGKAN (SEQ ID NO: 5)
Anti-myostatin adnectin DE loop:
PGRGVT (SEQ ID NO: 6)
Anti-myostatin adnectin FG loop:
TVTDTGYLKYKP (SEQ ID NO: 7)
Anti-myostatin adnectin core:
EVVAATPTSLLISWSLPHQGKANYYRITYGETGGNSPVQEFTVPGRGVTATISGLKPGVDYTIT
VYAVTVTDTGYLKYKPISINYRT (SEQ ID NO: 8)
Anti-myostatin adnectin core with N-terminal (AdNT1) (underlined) and
C-terminal (AdCT1) (italics) terminal sequence with His6 tag:
MGVSDVPRDLEVVAATPTSLLISWSLPHQGKANYYRITYGETGGNSPVQEFTVPGRGVTATISG
LKPGVDYTITVYAVTVIDTGYLKYKPISINYRTEIDKPSQHHHHHH (SEQ ID NO: 9)
Anti-myostatin adnectin core sequence preceded by N-terminal extension
sequence(GVSDVPRDL) and followed by a C-terminal tail (EI)):
GVSDVPRDLEVVAATPTSLLISWSLPHQGKANYYRITYGETGGNSPVQEFTVPGRGVTATISGL
KPGVDYTITVYAVTVTDTGYLKYKPISINYRTEI (SEQ ID NO: 10)
Anti-myostatin adnectin Fc-Fusion
DKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPELQLEESAAEAQEGELEGVSDVPRDLEVVAA
TPTSLLISWSLPHQGKANYYRITYGETGGNSPVQEFTVPGRGVTATISGLKPGVDYTITVYAVT
VTDTGYLKYKPISINYRTEI (SEQ ID NO: 78)
TGCGTGGTGGTGGACGTGAGCCACGAAGACCCTGAGGTCAAGTTCAACTGGTACGTGGACGGCG
TGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCAAGGTCTCC
AACAAAGCCCTCCCAGCCCCCATCGAGAAAACCATCTCCAAAGCCAAAGGGCAGCCCCGAGAAC
CACAGGTGTACACCCTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCCGAGCTGCAGCTGGAGGAAAGC
GCCGCTGAGGCTCAGGAAGGAGAACTGGAAGGCGTGAGCGACGTGCCACGGGATCTAGAAGTGG
TGGCTGCTACCCCCACAAGCTTGCTGATCAGCTGGTCTCTGCCGCACCAAGGTAAAGCCAATTA
TTACCGCATCACTTACGGCGAAACAGGAGGCAATAGCCCTGTCCAGGAGTTCACTGTGCCTGGT
CGTGGTGTTACAGCTACCATCAGCGGCCTTAAACCTGGCGTTGATTATACCATCACTGTGTATG
CTGTCACTGTTACTGATACAGGGTACCTCAAGTACAAACCAATTTCCATTAATTACCGGACCGA
AATT (SEQ ID NO: 82)
Anti-myostatin adnectin Fc-Fusion
GVSDVPRDLEVVAATPTSLLISWSLPHQGKANYYRITYGETGGNSPVQEFTVPGRGVTATISGL
KPGVDYTITVYAVTVTDTGYLKYKPISINYRTEIEPKSSDKTHTCPPCPAPELLGGPSVFLFPP
KPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHNHYT
QKSLSLSPGK (SEQ ID NO: 79)
GGCGTGAGCGACGTGCCCCGGGATCTAGAAGTGGTGGCTGCTACCCCCACAAGCTTGCTGATCA
GCTGGTCTCTGCCGCACCAAGGTAAAGCCAATTATTACCGCATCACTTACGGCGAAACAGGAGG
CAATAGCCCTGTCCAGGAGTTCACTGTGCCTGGTCGTGGTGTTACAGCTACCATCAGCGGCCTT
AAACCTGGCGTTGATTATACCATCACTGTGTATGCTGTCACTGTTACTGATACAGGGTACCTCA
AGTACAAACCAATTTCCATTAATTACCGGACCGAAATTGAGCCTAAGAGCTCCGACAAAACCCA
CACATGCCCACCTTGTCCAGCCCCCGAACTGCTGGGCGGCCCTTCAGTCTTCCTCTTCCCCCCA
AAACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCCTGCC
CCCATCCCGGGATGAGCTGACCAAGAACCAGGTCAGCCTGACCTGCCTGGTCAAAGGCTTCTAT
CCCAGCGACATCGCCGTGGAGTGGGAGAGCAATGGGCAGCCGGAGAACAACTACAAGACCACGC
CTCCCGTGTTGGACTCCGACGGCTCCTTCTTCCTCTACAGCAAGCTCACCGTGGACAAGAGCAG
GTGGCAGCAGGGGAACGTCTTCTCATGCTCCGTGATGCATGAGGCTCTGCACAACCACTACACG
CAGAAGAGCCTCTCCCTGTCTCCCGGGAAA (SEQ ID NO: 83)

1. A stable pharmaceutical formulation comprising
(i) at least 10 mg/mL of a polypeptide comprising a fibronectin type III tenth (¹⁰Fn3) domain which binds to myostatin;
(ii) a disaccharide at a concentration of at least 5%;
(iii) a histidine buffer at a concentration of between about 20 to about 60 mM; and
(iv) a pharmaceutically acceptable aqueous carrier,
wherein the formulation has a pH range of about 6.5 to about 7.8.

2. The formulation of claim 1, wherein the protein concentration of the anti-myostatin adnectin in the formulation is between about 10 mg/mL and 200 mg/mL, between about 10 mg/mL and 150 mg/mL, or between about 10 mg/mL and 85 mg/mL.

3. The formulation of claim 1, wherein the disaccharide is present at weight (w/w) ratio of at least 5:1 protein to sugar.

4. The formulation of claim 1, wherein the formulation comprises about 5% to about 30% of the disaccharide

5. The formulation of claim 1, wherein the concentration of the disaccharide is about 150 mM to about 800 mM, or about 300 to about 700 mM.

6. The formulation of claim 1, wherein the disaccharide is trehalose, and the formulation comprises about 5 to about 30% trehalose, about 15% to about 25% trehalose, or about 20% to about 25% trehalose.

7. The formulation of claim 1, wherein the disaccharide is trehalose dehydrate, and the concentration of trehalose dihydrate in the formulation is about 150 mM to about 800 mM, about 300 to about 700 mM, about 150 mM, about 200 mM, about 250 mM, about 300 mM, about 350 mM, about 400 mM, about 450 mM, about 500 mM, about 550 mM, about 575 mM, about 600, about 625 mM, about 650 mM, about 675 mM or about 700 mM.

8. The formulation of claim 1, wherein the histidine is present at a concentration of at least 20 mM.

9. The formulation of claim 1, wherein the viscosity of the formulation is from about 5 to 20 cps, from about 5 to 15 cps, or from about 7 to 12 cps.

10. The formulation of claim 1, wherein the pH is about 6.6 to 7.6, about 6.8 to 7.4, or about 7.0 to 7.3.

11. The formulation of claim 1, comprising a surfactant at a concentration of between about 0.01% and 0.5%.

12. The formulation of claim 1, comprising a chelator, wherein the concentration of the chelator is between about 0.01 mM and about 0.5 mM or between about 0.05 mM and 0.2 mM, and wherein the chelator is selected from the group consisting of DPTA, EDTA and EGTA.

13. A stable pharmaceutical formulation comprising,
(a) about 10-140 mg/mL of a polypeptide comprising a fibronectin type III tenth (¹⁰Fn3) domain which binds to myostatin;
about 5-25% trehalose dihydrate;
about 20-30 mM histidine; and
a pharmaceutically acceptable aqueous carrier,
wherein the pH of the formulation is about 6.8 to 7.3;
(b) about 10-140 mg/mL of a polypeptide comprising a fibronectin type III tenth (¹⁰Fn3) domain which binds to myostatin;
about 5-25% trehalose dihydrate;
about 20-30 mM histidine;
about 0.02-0.06 mM DTPA;
about 0.01-0.05% polysorbate 80; and
a pharmaceutically acceptable aqueous carrier,
wherein the pH of the formulation is about 6.8 to 7.3;
(c) about 10-140 mg/mL of a polypeptide comprising a fibronectin type III tenth (¹⁰Fn3) domain which binds to myostatin;
about 600 mM trehalose dihydrate;
25-30 mM histidine; and
a pharmaceutically acceptable aqueous carrier,
wherein the pH of the formulation is about 7.0 to 7.3;
(d) about 10-140 mg/mL of a polypeptide comprising a fibronectin type III tenth (¹⁰Fn3) domain which binds to myostatin;
about 600 mM trehalose dihydrate;
25-30 mM histidine;
about 0.02-0.06 mM DTPA;
about 0.01-0.05% polysorbate 80; and
a pharmaceutically acceptable aqueous carrier,
wherein the pH of the formulation is about 7.0 to 7.3;
(e) about 10-75 mg/mL of a polypeptide comprising a fibronectin type III tenth (¹⁰Fn3) domain which binds to myostatin;
about 5-25% trehalose dihydrate;
about 20-30 mM histidine; and
a pharmaceutically acceptable aqueous carrier,
wherein the pH of the formulation is about 6.8 to 7.3;
(f) about 10-75 mg/mL of a polypeptide comprising a fibronectin type III tenth (¹⁰Fn3) domain which binds to myostatin;
about 5-25% trehalose dihydrate;
about 20-30 mM histidine;
about 0.02-0.06 mM DTPA;
about 0.01-0.05% polysorbate 80; and
a pharmaceutically acceptable aqueous carrier,
wherein the pH of the formulation is about 6.8 to 7.3;
(g) about 10-75 mg/mL of a polypeptide comprising a fibronectin type III tenth (¹⁰Fn3) domain which binds to myostatin;
about 600 mM trehalose dihydrate;
about 30 mM histidine;
about 0.05 mM DTPA;
about 0.02% polysorbate 80;
a pharmaceutically acceptable aqueous carrier,
wherein the pH of the formulation is about 7.1.

14. A unit dosage form comprising about 1.0 mL or less of a formulation comprising,
(i) about 10-75 mg/mL of a polypeptide comprising a fibronectin type III tenth (¹⁰Fn3) domain which binds to myostatin;
(ii) about 5-25% trehalose dihydrate;
(iii) about 20-30 mM histidine;
(iv) about 0.02-0.06 mM DTPA;
(v) about 0.01-0.05% polysorbate 80; and
(vi) a pharmaceutically acceptable aqueous carrier,
wherein the pH of the formulation is about 6.8 to 7.3.

15. The formulation of claim 1, wherein at least one loop of the BC, DE, and FG loops of the ¹⁰Fn3 domain has 0, 1, 2, or 3 amino acid substitutions relative to the respective BC, DE, and FG loops of SEQ ID NOs: 5, 6 and 7, respectively.

16. The formulation of claim 1, wherein the ¹⁰Fn3 domain comprises the amino acid sequence of SEQ ID NO: 8.

17. The formulation of claim 1, wherein the polypeptide in the formulation comprises the amino acid sequence of SEQ ID NO: 78.

18. A method of attenuating or inhibiting a myostatin-related disease or disorder in a subject comprising administering an effective amount of a pharmaceutical formulation of claim 1, wherein the myostatin-related disease or disorder is a Amyotrophic Lateral Sclerosis (ALS), Becker's Muscular Dystrophy (BMD), Spinal Muscular Atrophy, Duchenne Muscular Dystrophy (DMD), sarcopenia or type II diabetes.

19. The method of claim 18, wherein the polypeptide comprising a fibronectin type III tenth (¹⁰Fn3) domain which binds to myostatin is administered at a dosage of about 5 mg to 200 mg, a dosage of about 5 mg to about 50 mg, a dosage of about 7.5 mg, about 15 mg, about 35 mg, or about 50 mg.

20. The method of claim 18 or 19, wherein the subject is a pediatric patient less than about 45 kg and is administered a dosage of about 7.5 mg to about 35 mg, or is more than about 45 kg and is administered a dosage of about 15 mg to about 50 mg.


SEQUENCE LISTING (83 sequences)

SEQ ID NO: 1 - protein, 375 aa, Homo sapiens
  misc_feature (1)..(375): Human prepromyostatin
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 352 aa, Homo sapiens [sequence omitted]

SEQ ID NO: 3 - protein, 109 aa, Unknown
  Mature myostatin conserved in human, murine, rat, chicken, turkey, dog, horse, and pig
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 4 - protein, 94 aa, Homo sapiens
  misc_feature (1)..(94): Wild-type human fibronectin type III domain (10Fn3)
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 5 - protein, 11 aa, Artificial Sequence
  Synthetic Anti-myostatin adnectin BC loop
SWSLPHQGKAN

SEQ ID NO: 6 - protein, 6 aa, Artificial Sequence
  Synthetic Anti-myostatin adnectin DE loop
PGRGVT

SEQ ID NO: 7 - protein, 12 aa, Artificial Sequence
  Synthetic Anti-myostatin adnectin FG loop
TVTDTGYLKYKP

SEQ ID NO: 8 - protein, 87 aa, Artificial Sequence
  Synthetic Anti-myostatin adnectin core
EVVAATPTSLLISWSLPHQGKANYYRITYGETGGNSPVQEFTVPGRGVTATISGLKPGVDYTITVYAVTVTDTGYLKYKPISINYRT

SEQ ID NO: 9 - protein, 110 aa, Artificial Sequence
  Synthetic Anti-myostatin adnectin core with N-terminal (AdNT1) (underlined) and C-terminal (AdCT1) (italics) terminal sequence with His6 tag
MGVSDVPRDLEVVAATPTSLLISWSLPHQGKANYYRITYGETGGNSPVQEFTVPGRGVTATISGLKPGVDYTITVYAVTVTDTGYLKYKPISINYRTEIDKPSQHHHHHH

SEQ ID NO: 10 - protein, 98 aa, Artificial Sequence
  Synthetic Anti-myostatin adnectin core sequence preceded by N-terminal extension sequence(GVSDVPRDL) and followed by a C-terminal tail (EI))
GVSDVPRDLEVVAATPTSLLISWSLPHQGKANYYRITYGETGGNSPVQEFTVPGRGVTATISGLKPGVDYTITVYAVTVTDTGYLKYKPISINYRTEI

SEQ ID NO: 11 - protein, 10 aa, Artificial Sequence
  Synthetic Exemplary leader, AdNT1
MGVSDVPRDL

SEQ ID NO: 12 - protein, 9 aa, Artificial Sequence
  Synthetic Exemplary leader, AdNT2
GVSDVPRDL

SEQ ID NO: 13 - protein, 8 aa, Artificial Sequence
  Synthetic Exemplary leader, AdNT3
VSDVPRDL

SEQ ID NO: 14 - protein, 7 aa, Artificial Sequence
  Synthetic Exemplary leader, AdNT4
SDVPRDL

SEQ ID NO: 15 - protein, 6 aa, Artificial Sequence
  Synthetic Exemplary leader, AdNT5
DVPRDL

SEQ ID NO: 16 - protein, 5 aa, Artificial Sequence
  Synthetic Exemplary leader, AdNT6
VPRDL

SEQ ID NO: 17 - protein, 4 aa, Artificial Sequence
  Synthetic Exemplary leader, AdNT7
PRDL

SEQ ID NO: 18 - protein, 3 aa, Artificial Sequence
  Synthetic Exemplary leader, AdNT8
RDL

SEQ ID NO: 19 - protein, 2 aa, Artificial Sequence
  Synthetic Exemplary leader, AdNT9
DL

SEQ ID NO: 20 - protein, 7 aa, Artificial Sequence
  Synthetic Exemplary tail, AdCT1
EIDKPSQ

SEQ ID NO: 21 - protein, 2 aa, Artificial Sequence
  Synthetic Exemplary tail, AdCT2
EI

SEQ ID NO: 22 - protein, 7 aa, Artificial Sequence
  Synthetic Exemplary tail, AdCT3
EIEPKSS

SEQ ID NO: 23 - protein, 6 aa, Artificial Sequence
  Synthetic Exemplary tail, AdCT4
EIDKPC

SEQ ID NO: 24 - protein, 5 aa, Artificial Sequence
  Synthetic Exemplary tail, AdCT5
EIDKP

SEQ ID NO: 25 - protein, 4 aa, Artificial Sequence
  Synthetic Exemplary tail, AdCT6
EIDK

SEQ ID NO: 26 - protein, 6 aa, Artificial Sequence
  Synthetic Exemplary tail, AdCT7
EIDKPS

SEQ ID NO: 27 - protein, 7 aa, Artificial Sequence
  Synthetic Exemplary tail, AdCT8
EIEKPSQ

SEQ ID NO: 28 - protein, 9 aa, Artificial Sequence
  Synthetic Exemplary tail, AdCT9
EIDKPSQLE

SEQ ID NO: 29 - protein, 12 aa, Artificial Sequence
  Synthetic Exemplary tail, AdCT10
EIEDEDEDEDED

SEQ ID NO: 30 - protein, 5 aa, Artificial Sequence
  Synthetic Exemplary tail, AdCT11
EGSGS

SEQ ID NO: 31 - protein, 7 aa, Artificial Sequence
  Synthetic Exemplary tail, AdCT12
EIDKPCQ

SEQ ID NO: 32 - protein, 4 aa, Artificial Sequence
  Synthetic Exemplary tail, AdCT13
GSGC

SEQ ID NO: 33 - protein, 5 aa, Artificial Sequence
  Synthetic Exemplary tail, AdCT14
EGSGC

SEQ ID NO: 34 - protein, 9 aa, Artificial Sequence
  Synthetic Exemplary tail, AdCT15
EIDKPCQLE

SEQ ID NO: 35 - protein, 13 aa, Artificial Sequence
  Synthetic Exemplary tail, AdCT16
EIDKPSQHHHHHH

SEQ ID NO: 36 - protein, 10 aa, Artificial Sequence
  Synthetic Exemplary tail, AdCT17
GSGCHHHHHH

SEQ ID NO: 37 - protein, 11 aa, Artificial Sequence
  Synthetic Exemplary tail, AdCT18
EGSGCHHHHHH

SEQ ID NO: 38 - protein, 6 aa, Artificial Sequence
  Synthetic Tag, T1
HHHHHH

SEQ ID NO: 39 - protein, 227 aa, Homo sapiens
  misc_feature (1)..(227): human IgG1 immunoglobulin Fc domain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 8 aa, Artificial Sequence
  Synthetic linker sequence
GAGGGGSG

SEQ ID NO: 41 - protein, 6 aa, Artificial Sequence
  Synthetic linker sequence
EPKSSD

SEQ ID NO: 42 - protein, 21 aa, Artificial Sequence
  Synthetic linker sequence
ESPKAQASSVPTAQPQAEGLA

SEQ ID NO: 43 - protein, 17 aa, Artificial Sequence
  Synthetic linker sequence
ELQLEESAAEAQDGELD

SEQ ID NO: 44 - protein, 9 aa, Artificial Sequence
  Synthetic linker sequence
GQPDEPGGS

SEQ ID NO: 45 - protein, 13 aa, Artificial Sequence
  Synthetic linker sequence
GGSGSGSGSGSGS

SEQ ID NO: 46 - protein, 17 aa, Artificial Sequence
  Synthetic linker sequence
ELQLEESAAEAQEGELE

SEQ ID NO: 47 - protein, 5 aa, Artificial Sequence
  Synthetic linker sequence
GSGSG

SEQ ID NO: 48 - protein, 4 aa, Artificial Sequence
  Synthetic linker sequence
GSGC

SEQ ID NO: 49 - protein, 7 aa, Artificial Sequence
  Synthetic linker sequence
AGGGGSG

SEQ ID NO: 50 - protein, 4 aa, Artificial Sequence
  Synthetic linker sequence
GSGS

SEQ ID NO: 51 - protein, 8 aa, Artificial Sequence
  Synthetic linker sequence
QPDEPGGS

SEQ ID NO: 52 - protein, 6 aa, Artificial Sequence
  Synthetic linker sequence
GSGSGS

SEQ ID NO: 53 - protein, 6 aa, Artificial Sequence
  Synthetic linker sequence
TVAAPS

SEQ ID NO: 54 - protein, 8 aa, Artificial Sequence
  Synthetic linker sequence
KAGGGGSG

SEQ ID NO: 55 - protein, 13 aa, Artificial Sequence
  Synthetic linker sequence
KGSGSGSGSGSGS

SEQ ID NO: 56 - protein, 9 aa, Artificial Sequence
  Synthetic linker sequence
KQPDEPGGS

SEQ ID NO: 57 - protein, 18 aa, Artificial Sequence
  Synthetic linker sequence
KELQLEESAAEAQDGELD

SEQ ID NO: 58 - protein, 7 aa, Artificial Sequence
  Synthetic linker sequence
KTVAAPS

SEQ ID NO: 59 - protein, 9 aa, Artificial Sequence
  Synthetic linker sequence
KAGGGGSGG

SEQ ID NO: 60 - protein, 14 aa, Artificial Sequence
  Synthetic linker sequence
KGSGSGSGSGSGSG

SEQ ID NO: 61 - protein, 10 aa, Artificial Sequence
  Synthetic linker sequence
KQPDEPGGSG

SEQ ID NO: 62 - protein, 19 aa, Artificial Sequence
  Synthetic linker sequence
KELQLEESAAEAQDGELDG

SEQ ID NO: 63 - protein, 8 aa, Artificial Sequence
  Synthetic linker sequence
KTVAAPSG

SEQ ID NO: 64 - protein, 8 aa, Artificial Sequence
  Synthetic linker sequence
AGGGGSGG

SEQ ID NO: 65 - protein, 7 aa, Artificial Sequence
  Synthetic linker sequence
AGGGGSG

SEQ ID NO: 66 - protein, 13 aa, Artificial Sequence
  Synthetic linker sequence
GSGSGSGSGSGSG

SEQ ID NO: 67 - protein, 9 aa, Artificial Sequence
  Synthetic linker sequence
QPDEPGGSG

SEQ ID NO: 68 - protein, 7 aa, Artificial Sequence
  Synthetic linker sequence
TVAAPSG

SEQ ID NO: 69 - protein, 16 aa, Artificial Sequence
  Synthetic hinge sequence
DKTHTCPPCPAPELLG

SEQ ID NO: 70 - protein, 16 aa, Artificial Sequence
  Synthetic hinge sequence
GSTHTCPPCPAPELLG

SEQ ID NO: 71 - protein, 24 aa, Artificial Sequence
  Synthetic hinge sequence
EPKSSDKTHTCPPCPAPELLGGPS

SEQ ID NO: 72 - protein, 24 aa, Artificial Sequence
  Synthetic hinge sequence
EPKSSDKTHTCPPCPAPELLGGSS

SEQ ID NO: 73 - protein, 24 aa, Artificial Sequence
  Synthetic hinge sequence
EPKSSGSTHTCPPCPAPELLGGSS

SEQ ID NO: 74 - protein, 19 aa, Artificial Sequence
  Synthetic hinge sequence
DKTHTCPPCPAPELLGGPS

SEQ ID NO: 75 - protein, 19 aa, Artificial Sequence
  Synthetic hinge sequence
DKTHTCPPCPAPELLGGSS

SEQ ID NO: 76 - protein, 330 aa, Homo sapiens [sequence omitted]

SEQ ID NO: 77 - protein, 20 aa, Artificial Sequence
  Synthetic leader sequence
METDTLLLWVLLLWVPGSTG

SEQ ID NO: 78 - protein, 340 aa, Artificial Sequence
  Synthetic Anti-myostatin adnectin Fc-Fusion
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESAAEAQEGELEGVSDVPRDLEVVAATPTSLLISWSLPHQGKANYYRITYGETGGNSPVQEFTVPGRGVTATISGLKPGVDYTITVYAVTVTDTGYLKYKPISINYRTEI

SEQ ID NO: 79 - protein, 330 aa, Artificial Sequence
  Synthetic Anti-myostatin adnectin Fc-Fusion
GVSDVPRDLEVVAATPTSLLISWSLPHQGKANYYRITYGETGGNSPVQEFTVPGRGVTATISGLKPGVDYTITVYAVTVTDTGYLKYKPISINYRTEIEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 206 aa, Homo sapiens
  misc_feature (1)..(206): human IgG1 CH2 and CH3 region
VFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 81 - protein, 20 aa, Artificial Sequence
  Synthetic exemplary N-terminal leader sequence for production of polypeptides in a mammalian system
METDTLLLWVLLLWVPGSTG

SEQ ID NO: 82 - DNA, 900 bp, Artificial Sequence
  Synthetic Anti-myostatin adnectin Fc-Fusion
tgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgagctgcagctggaggaaagcgccgctgaggctcaggaaggagaactggaaggcgtgagcgacgtgccacgggatctagaagtggtggctgctacccccacaagcttgctgatcagctggtctctgccgcaccaaggtaaagccaattattaccgcatcacttacggcgaaacaggaggcaatagccctgtccaggagttcactgtgcctggtcgtggtgttacagctaccatcagcggccttaaacctggcgttgattataccatcactgtgtatgctgtcactgttactgatacagggtacctcaagtacaaaccaatttccattaattaccggaccgaaatt

SEQ ID NO: 83 - DNA, 990 bp, Artificial Sequence
  Synthetic Anti-myostatin adnectin Fc-Fusion
ggcgtgagcgacgtgccccgggatctagaagtggtggctgctacccccacaagcttgctgatcagctggtctctgccgcaccaaggtaaagccaattattaccgcatcacttacggcgaaacaggaggcaatagccctgtccaggagttcactgtgcctggtcgtggtgttacagctaccatcagcggccttaaacctggcgttgattataccatcactgtgtatgctgtcactgttactgatacagggtacctcaagtacaaaccaatttccattaattaccggaccgaaattgagcctaagagctccgacaaaacccacacatgcccaccttgtccagcccccgaactgctgggcggcccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaa